(19) United States
(12) Patent Application Publication
(10) Pub. No.: US 2021/0008172 A1
(43) Pub. Date: Jan. 14, 2021

(54) LONG-ACTING IGF-1 OR IGF-1 VARIANTS AND METHODS OF PRODUCING SAME

(71) Applicant: OPKO Biologics Ltd., Kiryat Gat (IL)

(72) Inventors: OREN HERSHKOVITZ, M.P. Shikmim (IL); Lital Israeli-Yagev, Rehovot (IL); Ahuva Bar-Ilan, Rehovot (IL); Laura Moschcovich, Givat Shmuel (IL); Moran Golan, Kibbutz Shoval (IL)

(73) Assignee: OPKO Biologics Ltd., Kiryat Gat (IL)

(21) Appl. No.: 16/924,810

(22) Filed: Jul. 9, 2020

Related U.S. Application Data
(60) Provisional application No. 62/872,925, filed on Jul. 11, 2019.

(51) Int. Cl. A61K 38/30; C07K 1/107; C12N 15/64; C12N 15/69; A61K 38/27; A61K 38/17

(52) U.S. Cl. CPC A61K 38/30; C07K 1/1072; C12N 15/64; C12N 15/69; A61K 9/0019; A61K 38/1754; C07K 2319/02; C07K 2319/91; A61K 38/27

(57) ABSTRACT

Compositions which include polypeptides comprising at least one carboxy-terminal peptide (CTP) of chorionic gonadotropin attached to the carboxy terminus or amino terminus of an insulin-like growth factor 1 (IGF-1) or IGF-1 variant. Polynucleotides encoding the same are disclosed. Pharmaceutical compositions and pharmaceutical formulations comprising the polypeptides and polynucleotides of the invention and methods of using and producing same are also disclosed.

### CROSS REFERENCE TO RELATED APPLICATIONS

This application claims the benefit of U.S. Provisional Application Ser. No. 62/872,925, filed Jul. 11, 2019. This application is hereby incorporated by reference in its entirety herein.

### FIELD OF INVENTION

Compositions which include polypeptides comprising at least one carboxy-terminal peptide (CTP) of chorionic gonadotropin attached to the carboxy terminus or amino terminus of an insulin-like growth factor 1 (IGF-1) or IGF-1 variant. Polynucleotides encoding the same are disclosed. Pharmaceutical compositions and pharmaceutical formulations comprising the polypeptides and polynucleotides of the invention and methods of using and producing same are also disclosed.

### BACKGROUND OF THE INVENTION

Insulin-like growth factor-1, a somatomedin, is a small protein that has been shown to stimulate growth of a wide range of mammalian cells in culture. Human IGF-1(“hIGF-1” or “IGF-1”) has been purified to homogeneity from human serum and its complete amino acid sequence established. The serum mediator of growth hormone action, somatomedin C, has been shown to have an identical sequence to IGF-1 so that these two are now considered as being synonymous.

IGF-1 consists of 70 amino acids in a single chain with three s-s bridges and a MW of 7,649 Da. The amino acid sequence established for IGF-1 beginning with the N-terminal glycine is: Gly-Pro-Glu-Thr-Leu-Cys-Gly-Ala-Glu-Leu-Val-Asp-Ala-Leu-Gln-Phe-Val-Cys-Gly-Asp-Arg-Gly-Phe-Tyr-Phe-Asn-Lys-Pro-Thr-Gly-Tyr-Gly-Ser-Ser-Ser-Arg-Arg-Ala-Pro-Gln-Thr-Gly-Ile-Val-Asp-Glu-Cys-Cys-Phe-Arg-Ser-Cys-Asp-Leu-Arg-Arg-Leu- Glu-Met-.Tyr-Cys-Ala-Pro-Leu-Lys-Pro-Ala-Lys-Ser-Ala (SEQ ID NO: 1).

IGF-1 is highly homologous (47%) with human insulin and has 67% sequence identity with human IGF-2. Synthesis and release of both IGF-1 and IGF-2 is induced by growth hormone (“GH”). Most of the growth-promoting effects of human growth hormone are mediated through IGF-1, which bind to one specific receptor, IGFR1.

In blood circulation, almost all the IGF-1 is bound to carrier proteins called Insulin-like Growth Factor Binding Proteins (IGFBPs), from which IGFBP3 is the most important carrier for IGF-1, and to acid-labile subunit (ALS), forming a ternary complex with a molecular weight of 150 kDa. Formation of the ternary complexes restricts IGF-1 to the circulation, prolongs its half-lives and allows it to be stored at high concentration in plasma to facilitate its endocrine actions and to minimize its local effects due to its insulin-like activities such as hypoglycemia.

IGF-1 is a peptide hormone promotes systemic body growth inmost cells of the body. It is a primary mediator of growth hormone (GH), leading to statural growth: IGF-1 stimulate the uptake of glucose, fatty acids, and amino acids, which lead to cell, tissue, organ, and skeletal growth.

IGF-1 naturally occurs inhuman body fluids, for example, blood and human cerebral spinal fluid. Most tissues and especially the liver produce IGF-1 together with specific IGF-binding proteins. These molecules are under the control of growth hormone (GH). Like GH, IGF-1 is a potent anabolic protein. See Tanner et al., Acta Endocrinol., 84: 681-696 (1977); Uthne et al., J. Clin. Endocrinol. Metab., 39: 548-554 (1974)). IGF-1 has been isolated from human serum and produced recombinantly. See, e.g., EP 123,228 and 128,733.

It is generally accepted that distinct epitopes on IGF-I are used to bind receptor and binding proteins. It has been demonstrated in animal models that receptor-inactive IGF mutants are able to displace endogenous IGF-I from binding proteins and hereby generate a net IGF-I effect in vivo (Loddick et al., Proc. Natl. Acad. Sci. USA, 95: 1894-1898 (1998); Lowman et al., Biochemistry, 37: 8870-8878 (1998)). While residues Y24, Y29, Y31, and Y60 are implicated in receptor binding, IGF mutants thereof still bind to IGFBPs (Bayne et al., J. Biol. Chem. 265: 15648-15652 (1990); Bayne et al., J. Biol. Chem. 264: 11004-11008 (1989); Cascieri et al., Biochemistry, 27: 3229-3233 (1988); Lowman et al., supra.

Additionally, a variant designated (1-27,gly4,38-70)-hIGF-1, wherein residues 28-37 of the C region of human IGF-1 are replaced by a four-residue glycine bridge, has been discovered that binds to IGFBP's but not to IGF receptors (Bar et al., Endocrinology, 127: 3243-3245 (1990)).

A multitude of mutagenesis studies have addressed the characterization of the IGFBP-binding epitope on IGF-I (Bagley et al., Biochem. J., 259: 665-671 (1989); Baxter et al., J. Biol. Chem. 267: 60-65 (1992); Bayne et al., J. Biol. Chem. 263: 6233-6239 (1988); Clemmons et al., J. Biol. Chem., 265: 12210-12216 (1990); Clemmons et al., Endocrinology, 131: 890-895 (1992); Oh et al., supra). In summary, the N-terminal residues 3 and 4 and the helical region comprising residues 8-17 were found to be important for binding to the IGFBP's. Additionally, an epitope involving residues 49-51 in binding to IGFBP-1, -2 and -5 has been identified (Clemmons et al., Endocrinology, supra, 1992). Furthermore, a naturally occurring truncated form of IGF-I lacking the first three N-terminal amino acids (called des(1-3)-IGF-I) was demonstrated to bind IGFBP-3 with 25 times lower affinity (Heding et al., J. Biol. Chem. 271: 13948-13952 (1996); U.S. Pat. Nos. 5,077,276; 5,164,370; 5,470,828).

In an attempt to characterize the binding contributions of exposed amino acid residues in the N-terminal helix, several alanine mutants of IGF-I were constructed (Jansson et al., Biochemistry, 36: 4108-4117 (1997)). However, the circular dichroism spectra of these mutant proteins showed structural changes compared to wild-type IGF-I, making it difficult to clearly assign IGFBP-binding contributions to the mutated side chains. A different approach was taken in a very recent study where the IGFBP-1 binding epitope on IGF-I was probed by heteronuclear NMR spectroscopy (Jansson et al., J. Biol. Chem., 273: 24701-24707 (1998)). The authors additionally identified residues R36, R37 and R50 to be functionally involved in binding to IGFBP-1.

Other IGF-I variants have been disclosed. For example, in the patent literature, WO 96/33216 describes a truncated variant having residues 1-69 of authentic IGF-I. EP 742,228 discloses two-chain IGF-I superagonists which are derivatives of the naturally occurring single-chain IGF-I having an abbreviated C domain. The IGF-I analogs are of the formula: BCn, A wherein B is the B domain of IGF-I or a functional analog thereof, C is the C domain of IGF-I or a functional analog thereof, n is the number of amino acids in the C domain and is from about 6 to about 12, and A is the A domain of IGF-I or a functional analog thereof.

Additionally, Cascieri et al., Biochemistry 27: 3229-3233 (1988) discloses four mutants of IGF-I, three of which have reduced affinity to the Type 1 IGF receptor. These mutants are: (Phe23, Phe24, Tyr25)IGF-I (which is equipotent to human IGF-I in its affinity to the Types 1 and 2 IGF and insulin receptors), (Leu24)IGF-I and (Ser24)IGF-I (which have a lower affinity than IGF-I to the human placental Type 1 IGF receptor, the placental insulin receptor, and the Type 1 IGF receptor of rat and mouse cells), and desoctapeptide (Leu24)IGF-I (in which the loss of aromaticity at position 24 is combined with the deletion of the carboxyl-terminal D region of hIGF-I, which has lower affinity than (Leu24)IGF-I for the Type 1 receptor and higher affinity for the insulin receptor). These four mutants have normal affinities for human serum binding proteins.

Bayne et al., J. Biol. Chem., 264: 11004-11008 (1988) discloses three structural analogs of IGF-I: (1-62)IGF-1, which lacks the carboxyl-terminal 8-amino-acid D region of IGF-I; (1-27,Gly4,38-70)IGF-I, in which residues 28-37 of the C region of IGF-I are replaced by a four-residue glycine bridge; and (1-27,Gly4,38-62)IGF-I, with a C region glycine replacement and a D region deletion. Peterkofsky et al., Endocrinology, 128: 1769-1779 (1991) discloses data using the Gly4 mutant of Bayne et al., supra, Vol. 264. U.S. Pat. No. 5,714,460 refers to using IGF-I or a compound that increases the active concentration of IGF-I to treat neural damage.

Cascieri et al., J. Biol. Chem., 264: 2199-2202 (1989) discloses three IGF-I analogs in which specific residues in the A region of IGF-I are replaced with the corresponding residues in the A chain of insulin. The analogs are: (Ile41, Glu45, Gln46, Thr49, Ser50, Ile51, Ser53, Tyr55, Gln56)IGF-I, an A chain mutant in which residue 41 is changed from threonine to isoleucine and residues 42-56 of the A region are replaced; (Thr49, Ser50, Ile51)IGF-I; and (Tyr55, Gln56)IGF-I.

WO 94/04569 discloses a specific binding molecule, other than a natural IGFBP, that is capable of binding to IGF-I and can enhance the biological activity of IGF-I. WO98/45427 published Oct. 15, 1998 and Lowman et al., supra, disclose IGF-I agonists identified by phage display. Also, WO 97/39032 discloses ligand inhibitors of IGFBP's and methods for their use. Further, U.S. Pat. No. 5,891,722 discloses antibodies having binding affinity for free IGFBP-1 and devices and methods for detecting free IGFBP-1 and a rupture in a fetal membrane based on the presence of amniotic fluid in a vaginal secretion, as indicated by the presence of free IGFBP-1 in the vaginal secretion.

Various biological activities of IGF-1 have been identified. Researchers have found that an intravenous bolus injection of IGF-1 lowers blood glucose levels in humans. See Guler et al., N. Engl. J. Med., 317: 137-140 (1987). Additionally, IGF-1 promotes growth in several metabolic conditions characterized by low IGF-1 levels, such as hypophysectomized rats [Guler et al., Endocrinology, 118: Supp 129 abstract, Skottner et al., J. Endocr., 112: 123-132 (1987); Guler et al., Proc, Natl. Acad, Sci, USA, 85: 4889-4893 (1988); Froesch et al., in Endocrinology. Intl. Congress Series 655, ed. by Labrie and Proulx (Amsterdam: Excerpta Medica, 1984), p. 475-479], diabetic rats [Scheiwiller et al., Nature, 323: 169-171 (1986)], and dwarf rats [Skottner et al., Endocrinology, 124: 2519-2526 (1989)]. The kidney weight of hypophysectomized rats increases substantially upon prolonged infusions of IGF-1 subcutaneously. Guler et al., Proceedings of the 1st European Congress of Endocrinology, 103: abstract 12-390 (Copenhagen, 1987). The kidneys of Snell dwarf mice and dwarf rats behaved similarly. van Buul-Offers et al., Pediatr. Res., 20: 825-827 (1986); Skottner et al., Endocrinclogy, supra. An additional use for IGF-1 is its administration to improve glomerular filtration and renal plasma flow in human patients. See EP 327,503 published Aug. 9, 1989; Guler et al., Proc. Natl. Acad. Sci. USA, 86: 2868-2872 (1989).

Some dwarfism diseases, named severe primary IGF deficiency (SP IGFD) includes patients with mutations in the GH receptor (GHR), post-GHR signaling pathway, or IGF1 gene defects. These patients cannot respond to GH treatment and may be treated with IGF1. Commercial recombinant IGF1 (Mecasermin, brand name Increlex) is approved for the treatment for this growth failure, but twice daily subcutaneous injections are required. Commercial recombinant IGF-1 is also approved for treatment of growth failure in children who have developed neutralizing antibodies to growth hormone.

Due to an insulin-like hypoglycemic effect, Increlex should be administered shortly before or after a meal.

Accordingly, it is an object of an aspect of the present invention to overcome, or at least alleviate, one or more of the difficulties related to the prior art.

It is an object of the present invention to provide a long acting IGF-1 that has a longer half-life, and that is more efficient and more convenient than the drugs currently available in the market

It is another object of the present invention to conjugate the carboxy terminal peptide (CTP) of human chorionic gonadotropin, which is highly glycosylated. Proteins attached to this peptide are expected to have a slower clearance by the kidneys due to their charge, increased molecular weight and globular size. Proteins attached to this peptide are also expected to have a slower clearance by the liver due to its low affinity to asialoglycoprotein receptors. The CTP-modified IGF-1, conjugated to several copies of CTP, can reduce injections frequency, provide easier handling for the patients and an improved safety profile due to the lack of hypoglycemic effect, and hence significantly increase life quality of patients, including those with severe primary IGFD.

These and other objects will be apparent to those of ordinary skill in the art.

### SUMMARY OF THE INVENTION

In one aspect, disclosed is a polypeptide comprising a CTP-modified insulin-like growth factor 1 (IGF-1) or CTP-modified IGF-1 variant, said CTP-modified IGF-1 or IGF-1 variant comprising at least one chorionic gonadotrophin carboxy terminal peptide (CTP) attached to the amino terminus or carboxy terminus of said IGF-1 or IGF-1 variant. In another aspect, disclosed is a polypeptide comprising a CTP-modified insulin-like growth factor 1 (IGF-1) or IGF-1 variant, said CTP-modified IGF-1 comprising at between three to six chorionic gonadotrophin carboxy terminal peptides (CTPs) attached to the amino terminus or carboxy terminus of said IGF-1 or IGF-1 variant.

In one aspect, the present invention provides a CTP-modified insulin-like growth factor 1 (IGF-1) polypeptide wherein no chorionic gonadotrophin carboxy terminal peptides (CTPs) are attached to the amino terminus of said IGF-1, and three or four CTPs are attached to the carboxy terminus of said IGF-1, wherein the average EC₅₀ value of said Insulin receptor and said IGF-1 receptor (EC₅₀ Insulin receptor/EC₅₀ IGF-1 receptor) are present in a ratio of between 30 to 400.

In one aspect, the present invention provides a polynucleotide encoding the CTP-modified IGF-1 or IGF-1 variants disclosed herein.

In a further aspect, the present the present invention provides pharmaceutical compositions comprising the CTP-modified IGF-1 or IGF-1 variants disclosed herein.

In one aspect, the present invention provides methods of treating a human patient having an IGF-1 related disease or disorder comprising administering a pharmaceutically effective amount of the CTP-modified IGF-1 or IGF-1 variants disclosed herein.

In another aspect, the present invention provides a method of manufacturing the CTP-modified IGF-1 or IGF-1 variants disclosed herein, the method comprising the steps of (a) stably transfecting a predetermined number of cells with an expression vector comprising a coding portion encoding said CTP-modified IGF-1 or IGF-1 variant; (b) wherein said transfected cells express and secrete said CTP-modified IGF-1 or IGF-1 variant; (c) obtaining cell clones that overexpress said CTP-modified IGF-1 or IGF-1 variant; (d) expanding said clones in solution to a predetermined scale; (e) harvesting said solution containing said clones; (f) filtering said solution containing said clones to obtain a clarified harvest solution containing said CTP-modified IGF-1 or IGF-1 variant; and, (g) purifying and activating CTP-modified IGF-1 or IGF-1 variant from said clarified harvest solution to obtain a purified protein solution having a desired concentration of the CTP-modified IGF-1 or IGF-1 variant, thereby manufacturing a CTP-modified IGF-1 or IGF-1 variant.

In one aspect, the present invention provides a combination comprising a therapeutically effective amount of a CTP modified IGF-1 or variant thereof and a therapeutically effective amount of an active ingredient selected from the group consisting of human growth hormone (HGH) and IGF1 binding protein.

In one aspect, the present invention provides a therapeutic regimen comprising administering a therapeutically effective dose of a CTP modified IGF-1 or variant thereof in combination with a therapeutically effective amount of human growth hormone or in combination with an IGF1 binding protein or any combination thereof effective to treat an IGF-1 related disease, disorder or condition in a patient in need of treatment thereof

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the body weight gain (BWG) at WGA #4 of MOD-1301-2, MOD-1301-3, and MOD-1301-5.

FIG. 2 shows the average PK results of the CTP-modified IGF-1 variants vs. Increlex.

FIG. 3 shows the CBA results of IGF1 receptor activation by Increlex and MOD-1301 variants

FIG. 4 shows blood glucose levels (% from basal) following injection of Increlex or MOD-1301 variants.

FIG. 5 shows blood glucose levels following injection of Increlex or MOD-1301 variants.

### DETAILED DESCRIPTION OF THE PRESENT INVENTION

### IGF-1

In one embodiment “IGF-1” refers to insulin-like growth factor 1 from any species, including bovine, ovine, porcine and human, in native-sequence or variant form, including but not limited to naturally-occurring allelic variants. IGF-1 may be from any source, whether natural, synthetic or recombinant, provided that it will bind IGFBP-3 at the appropriate site. IGF-1 can be produced recombinantly, for example, as described in PCT publication WO 95/04076.

In one embodiment, the IGF-1 protein is a human IGF-1 protein.

In one embodiment, the IGF-1 protein is a recombinant human IGF-1 (rhIGF-1).

In one embodiment, the IGF-I variants are those described in U.S. Pat. Nos. 5,077,276; 5,164,370; or 5,470,828; or in WO 87/01038, i.e., those wherein at least the glutamic acid residue is absent at position 3 from the N-terminus of the mature molecule or those having a deletion of up to five amino acids at the N-terminus. The most preferred variant has the first three amino acids from the N-terminus deleted (variously designated as brain IGF, tIGF-I, des(1-3)-IGF-I, or des-IGF-I).

In one embodiment, the codon sequence of IGF-1 consist of the following four features, from N terminal to C terminal: signal peptide (SP), first pro-peptide (PP), IGF-1 chain sequence itself, which is the active unit, and a second pro-peptide, the E peptide. The features of IGF-1 are described in Table 1. The N-terminal pro-peptide is defined as the signal peptide (SP) plus the closest pro-peptide to the N-terminus (the “N-terminal pro-peptide” or positions 1-48) of human IGF-1.

TABLE 1

| Feature key | Position | Amino Acid Length |
|---|---|---|
| Signal peptide (SP) | 1-21 | 21 |
| First Pro-peptide (PP) | 22-48 | 27 |
| Chain (IGF-1) | 49-118 | 70 |
| Second Pro-peptide (E peptide) | 119-195 | 77 |

In another embodiment, the invention includes a homologue of IGF-1. In another embodiment, the invention includes a homologue of IGF-1. In another embodiment, homologues e.g., include polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous IGF-1 as determined using BlastP software of the National Center of Biotechnology Information (NCBI) using default parameters.

In another embodiment, disclosed are analogs of IGF-1. In one embodiment, the IGF-1 analogs have the same therapeutic effect as IGF-1 in humans or animals. In another embodiment, the IGF-1 analogs are naturally occurring analogs of IGF-I (e.g., truncated IGF-1) or any of the known synthetic analogs of IGF-1. See, for example, U.S. Pat. Nos. 6,251,865 and 5,473,054.

In one embodiment, the present invention provides a polypeptide comprising at least one carboxy-terminal peptide (CTP) of chorionic gonadotropin attached to the carboxy terminus or amino terminus of an insulin-like growth factor 1 (IGF-1).

In another embodiment, “signal sequence” and “signal peptide” are used interchangeably herein having all the same qualities and meanings. In another embodiment, “sequence” when in reference to a polynucleotide molecule can refer to a coding portion. In another embodiment, an engineered IGF-1 comprising at least one CTP as described herein has enhanced in vivo biological activity compared the same IGF-1 without at least one CTP. In one embodiment, the enhanced biological activity stems from the longer half-life of the engineered IGF-1 while maintaining at least some biological activity. In another embodiment, the enhanced biological activity stems from enhanced biological activity resulting from the CTP modification. In another embodiment, the enhanced biological activity stems from both a longer half-life and from enhanced functionality of the CTP-modified IGF-1.

In one embodiment, the CTP-modified IGF-1 includes a signal peptide. In another embodiment, the CTP-modified IGF-1 does not comprise a signal peptide.

In one embodiment, the amino acid signal peptide sequence of IGF-1 (“SPIGF1”) is MGKISSLPTQLFKCCFCDFLK (SEQ ID NO: 2).

In one embodiment, the amino acid sequence of the first propeptide of IGF-1 (“PP”) is VKMHTMSSSHLFYLALCLLTFTSSATA (SEQ ID NO: 3).

In one embodiment, the amino acid sequence of the N-terminal pro-peptide is MGKISSLPTQLFKCCFCDFLKVKMHTMSSSHLFYLALCLLTFTSSATA (SEQ ID NO: 25).

In one embodiment, the amino acid sequence of the IGF-1 chain (“Chain”) is GPETLCGAELVDALQFVCGDRGFYFNKPTGYGSSSRRAPQTGIVDECCFRSCDLRRLEM YCAPLKPAKSA (SEQ ID NO: 1).

In one embodiment, the amino acid sequence of the second propeptide of IGF-1 (“E peptide” or “EP”) is RSVRAQRHTDMPKTQKYQPPSTNKNTKSQRRKGWPKTHPGGEQKEGTEASLQIRGKK KEQRREIGSRNAECRGKKGK (SEQ ID NO: 4).

In one embodiment, the full sequence of IGF-1, including signal peptides and pro-peptides, is MGKISSLPTQLFKCCFCDFLKVKMHTMSSSHLFYLALCLLTFTSSATAGPETLCGAELV DALQFVCGDRGFYFNKPTGYGSSSRRAPQTGIVDECCFRSCDLRRLEMYCAPLKPAKSA RSVRAQRHTDMPKTQKYQPPSTNKNTKSQRRKGWPKTHPGGEQKEGTEASLQIRGKK KEQRREIGSRNAECRGKKGK (SEQ ID NO: 5).

In one embodiment, the term “insulin-like growth factor-1” or “IGF-1 peptide” or simply “IGF-1”, as used throughout the specification and in the claims, refers to a polypeptide product which exhibits similar, in-kind, biological activities to natural insulin-like growth factor-1, as measured in recognized bioassays, and has substantially the same amino acid sequence as native IGF-1. It will be understood that polypeptides deficient in one or more amino acids in the amino acid sequence reported in the literature for naturally occurring IGF-1, or polypeptides containing additional amino acids or polypeptides in which one or more amino acids in the amino acid sequence of natural IGF-1 are replaced by other amino acids are within the scope of the invention, provided that they exhibit the functional activity of IGF-1, e.g., by acting synergistically with other growth factors in accelerating the healing of soft and mesenchymal tissue wounds. The invention is intended to embrace all the allelic variations of IGF-1. Moreover, as noted above, derivatives obtained by simple modification of the amino acid sequence of the naturally occurring product, e.g, by way of site-directed mutagenesis or other standard procedures, are included within the scope of the present invention. Forms of IGF-1 produced by proteolysis of host cells that exhibit similar biological activities to mature, naturally occurring IGF-1 are also encompassed by the present invention.

As used herein, the term “long acting IGF-1” or “CTP-modified IGF-1” refers to either the CTP-modification of IGF-1 or an IGF-1 variant.

In one embodiment, the IGF-1 variant comprises an alanine, a glycine, or a serine substitution of the amino acid residue at position 16, 25, or 49 of native sequence human IGF-1, or an alanine, a glycine, or a serine substitution of the amino acid residues at positions 3 and 49 of native-sequence human IGF-1. In another embodiment, the IGF-1 variant comprises replacement of the amino acid residues at position 3 and at position 49 with alanine residues compared to the native human IGF-1 sequence.

In one embodiment, the IGF-1 variant comprises a replacement of an amino acid residue located at a single position selected from the group consisting of positions 4, 5, 7, 10, 14, 17, 23, 24, and 43 of native-sequence human IGF-I with an alanine residue.

In another embodiment, the IGF-1 variant comprises a replacement of an amino acid residue at positions 1 and 70 of native-sequence human IGF-I with a serine residue and a valine residue, respectively.

In another embodiment, the IGF-1 variant comprises a replacement of an amino acid residue at positions 1 and 70 of native-sequence human IGF-1 with a serine residue and a valine residue, respectively, and a replacement of an amino acid residue at a single position selected from the group consisting of positions 3, 4, 5, 7, 10, 14, 17, 23, 24, 25, and 43 of native-sequence human IGF-I with an alanine residue.

In another embodiment, “IGFBP-3” refers to insulin-like growth factor binding protein 3. IGFBP-3 is a member of the insulin-like growth factor binding protein family. IGFBP-3 may be from any species, including bovine, ovine, porcine and human, in native-sequence or variant form, including but not limited to naturally-occurring allelic variants. IGFBP-3 can form a binary complex with IGF-I, and a ternary complex with IGF and the acid labile subunit (ALS). IGFBP-3 may be from any source, whether natural, synthetic or recombinant, provided that it will bind IGF-I and ALS at the appropriate sites. IGFBP-3 can be produced recombinantly, as described in PCT publication WO 95/04076.

Chorionic Gonadotrophin Carboxy Terminal Peptides (cgCTPs)

In one embodiment, the present invention provides a polypeptide comprising an IGF-1 polypeptide or variant thereof and at least two chorionic gonadotrophin carboxy terminal peptides (cgCTPs).

A skilled artisan would appreciate that the terms “CTP peptide”, “CTP”, “human chorionic gonadotropin carboxy terminal peptide”, “hcgCTP”, “cgCTP”, “carboxy terminal peptide” and “CTP sequence” may be used interchangeably herein. In one embodiment, a carboxy terminal peptide is a full-length CTP. In another embodiment, the carboxy terminal peptide is a truncated CTP.

In one embodiment, the CTP sequence comprises: DPRFQDSSSSKAPPPSLPSPSRLPGPSDTPILQ (SEQ ID NO: 6). In another embodiment, the CTP sequence comprises: SSSSKAPPPSLPSPSRLPGPSDTPILPQ (SEQ ID NO: 7). In another embodiment, the CTP sequence comprises an amino acid sequence selected from the sequences set forth in SEQ ID NO: 6 and SEQ ID NO: 7. In another embodiment, the CTP sequence comprises a partial amino acid sequence selected from the SEQ ID NO: 6 or SEQ ID NO: 7.

In one embodiment, the carboxy terminal peptide (CTP) peptide of the present invention comprises the amino acid sequence from amino acid 112 to position 145 of human chorionic gonadotrophin. In another embodiment, the human chorionic gonadotrophin carboxy terminal peptide of the present is referred to as either CTP or cgCTP. In another embodiment, the CTP sequence of the present invention comprises the amino acid sequence from amino acid 118 to position 145 of human chorionic gonadotropin, as set forth in SEQ ID NO: 2. In another embodiment, the CTP sequence also commences from any position between positions 112-118 and terminates at position 145 of human chorionic gonadotrophin. In some embodiments, the CTP sequence peptide is 28, 29, 30, 31, 32, 33 or 34 amino acids long and commences at position 112, 113, 114, 115, 116, 117 or 118 of the CTP amino acid sequence.

In one embodiment, the cgCTP of the compositions and methods of the present invention is truncated. In one embodiment, the truncated CTP comprises SSSSKAPPPSLP (SEQ ID NO: 8). In another embodiment, the truncated CTP comprises the first 10 amino acids of SEQ ID NO: 8. In another embodiment, the truncated CTP comprises the first 11 amino acids of SEQ ID NO: 8.

In one embodiment, the truncated CTP comprises the first 15 amino acids of SEQ ID NO: 7. In one embodiment, the truncated CTP comprises the first 14 amino acids of SEQ ID NO: 7. In one embodiment, the truncated CTP comprises the first 13 amino acids of SEQ ID NO: 7. In one embodiment, the truncated CTP comprises the first 12 amino acids of SEQ ID NO: 7. In one embodiment, the truncated CTP comprises the first 11 amino acids of SEQ ID NO: 7. In one embodiment, the truncated CTP comprises the first 10 amino acids of SEQ ID NO: 7. In one embodiment, the truncated CTP comprises the first 9 amino acids of SEQ ID NO: 7. In one embodiment, the truncated CTP comprises the first 8 amino acids of SEQ ID NO: 7 or SEQ ID NO: 8. In one embodiment, the truncated CTP comprises the first 7 amino acids of SEQ ID NO: 7 or SEQ ID NO: 8. In one embodiment, the truncated CTP comprises the first 6 amino acids of SEQ ID NO: 7 or SEQ ID NO: 8. In one embodiment, the truncated CTP comprises the first 5 amino acids of SEQ ID NO: 7 or SEQ ID NO: 8.

In another embodiment, the CTP peptide is a variant of chorionic gonadotrophin CTP which differs from the native CTP by 1-5 conservative amino acid substitutions as described in U.S. Pat. No. 5,712,122, which is incorporated herein by reference in its entirety. In another embodiment, the CTP peptide is a variant of chorionic gonadotrophin CTP which differs from the native CTP by 1 conservative amino acid substitution. In another embodiment, the CTP peptide is a variant of chorionic gonadotrophin CTP which differs from the native CTP by 2 conservative amino acid substitutions. In another embodiment, the CTP peptide is a variant of chorionic gonadotrophin CTP which differs from the native CTP by 3 conservative amino acid substitutions. In another embodiment, the CTP peptide is a variant of chorionic gonadotrophin CTP which differs from the native CTP by 4 conservative amino acid substitutions. In another embodiment, the CTP peptide is a variant of chorionic gonadotrophin CTP which differs from the native CTP by 5 conservative amino acid substitutions.

In another embodiment, the CTP peptide amino acid sequence of the present invention is at least 70% homologous to the native CTP amino acid sequence or a peptide thereof. In another embodiment, the CTP peptide amino acid sequence of the present invention is at least 80% homologous to the native CTP amino acid sequence or a peptide thereof. In another embodiment, the CTP peptide amino acid sequence of the present invention is at least 85% homologous to the native CTP amino acid sequence or a peptide thereof. In another embodiment, the CTP peptide amino acid sequence of the present invention is at least 90% homologous to the native CTP amino acid sequence or a peptide thereof. In another embodiment, the CTP peptide amino acid sequence of the present invention is at least 95% homologous to the native CTP amino acid sequence or a peptide thereof. In another embodiment, the CTP peptide amino acid sequence of the present invention is at least 98% homologous to the native CTP amino acid sequence or a peptide thereof.

In one embodiment, the long acting IGF-1 comprises a single cgCTP attached to the amino terminus of said IGF-1. In another embodiment, the long acting IGF-1 comprises two cgCTPs at the amino terminus of said IGF-1. In another embodiment, the long acting IGF-1 comprises three cgCTPs at the amino terminus of said IGF-1. In another embodiment, the long acting IGF-1 comprises four cgCTPs at the amino terminus of said IGF-1. In another embodiment, the long acting IGF-1 comprises five cgCTPs at the amino terminus of said IGF-1. In another embodiment, the long acting IGF-1 comprises one to five cgCTPs at the amino terminus of said IGF-1 and no cgCTPs attached to the carboxy terminus.

In one embodiment, the long acting IGF-1 comprises a single cgCTP at the carboxy terminus of said IGF-1. In another embodiment, the long acting IGF-1 comprises two cgCTPs at the carboxy terminus of said IGF-1. In another embodiment, the long acting IGF-1 comprises three cgCTPs at the carboxy terminus of said IGF-1. In another embodiment, the long acting IGF-comprises four cgCTPs at the carboxy terminus of said IGF-1. In another embodiment, the long acting IGF-comprises five cgCTPs at the carboxy terminus of said IGF-1. In another embodiment, the long acting IGF-1 comprises one to five cgCTPs at the carboxy terminus of said IGF-1 and no cgCTPs attached to the amino terminus.

In one embodiment, the long acting IGF-1 comprises a single cgCTP attached to the amino terminus and a single cgCTP at the carboxy terminus. In another embodiment, the long acting IGF-1 comprises a single cgCTP at the amino terminus and two cgCTPs at the carboxy terminus. In another embodiment, the long acting IGF-1 comprises a single cgCTP at the amino terminus and three cgCTPs at the carboxy terminus. In another embodiment, the long acting IGF-1 comprises a single cgCTP at the amino terminus and four cgCTPs at the carboxy terminus. In another embodiment, the long acting IGF-1 comprises a single cgCTP at the amino terminus and five cgCTPs at the carboxy terminus.

In one embodiment, the long acting IGF-1 comprises a single cgCTP attached to the carboxy terminus and a single cgCTP at the amino terminus. In another embodiment, the long acting IGF-1 comprises a single cgCTP at the carboxy terminus and two cgCTPs at the amino terminus. In another embodiment, the long acting IGF-1 comprises a single cgCTP at the carboxy terminus and three cgCTPs at the amino terminus. In another embodiment, the long acting IGF-1 comprises a single cgCTP at the carboxy terminus and four cgCTPs at the amino terminus. In another embodiment, the long acting IGF-1 comprises a single cgCTP at the carboxy terminus and five cgCTPs at the amino terminus.

In one embodiment, the N terminal pro-peptide, which includes the signal peptide (SP) and the first pro-peptide (PP), is needed in order to allow IGF-1 secretion following their cleavage.

In one embodiment, the CTP-modified IGF-1 does not include the E peptide.

In one embodiment, the signal peptide of the IGF-1 construct is a signal peptide of a human growth hormone (“SPhGH”) and is present at the amino terminus of the CTP-modified IGF-1. In another embodiment, the first propeptide of IGF-1 follows the signal peptide at the amino terminus of the CTP-modified IGF-1 and is represented by the following structure, from N terminus to C terminus, SPhGH-PPIGF1.

In one embodiment, the signal peptide of the IGF-1 (SPIGF1) is present at the amino terminus of the CTP-modified IGF-1. In another embodiment, the first propeptide of IGF-1 follows the signal peptide at the amino terminus of the CTP-modified IGF-1 and is represented by the following structure, from N terminus to C terminus, SPIGF1-PPIGF1.

In another embodiment, the signal peptide of a human growth hormone (“SPhGH”) comprises the following amino acid sequence: MATGSRTSLLLAFGLLCLPWLQEGSA (SEQ ID NO: 9).

In various embodiments, the claimed constructs and polypeptides produced therefrom are shown or depicted structurally by the following embodiments. The IGF-1 polypeptide with modifications on the amino terminus are depicted on the left side of IGF-1 and the modifications on the carboxy terminus are depicted on the right side of the IGF-1 designation. In one embodiment, the CTP-modified IGF-1 has the structure, from N terminal to C terminal: SP-(CTP)1-5-IGF1. In another embodiment, the CTP-modified IGF-1 has the structure, from N terminal to C terminal: SP-IGF1-(CTP)1-5. In another embodiment, the CTP-modified IGF-1 has the structure, from N terminal to C terminal: SP-PPIGF1-IGF1-(CTP)1-5. In another embodiment, the CTP-modified IGF-1 has the structure, from N terminal to C terminal: SP-PPIGF1-(CTP)1-5-IGF1. In another embodiment, the CTP-modified IGF-1 has the structure, from N terminal to C terminal: SP-(CTP)1-2-IGF1-(CTP)1-5. In another embodiment, the CTP-modified IGF-1 has the structure, from N terminal to C terminal: SP-PPIGF1-(CTP)1-2-IGF1-(CTP)1-5. The SP in the CTP-modified IGF-1 structure can be the signal peptide of either IGF-1 or hGH or the SP in the CTP-modified IGF-1 structure can be any signal peptide. IGF-1 in these embodiments does not include the C terminus pro-peptide (the E peptide). IGF1 as shown below means any active IGF-1 polypeptide or variant thereof.

In another embodiment, only a signal peptide (SP) is needed in order to allow IGF-1 secretion following its cleavage. In another embodiment, the signal peptide necessary for secretion can be any signal peptide disclosed herein.

In one embodiment, the CTP-modified IGF-1 expressed construct and active polypeptides are described in Table 2.

TABLE 2
Five CTP-Modified IGF-1 Variants

| Expressed Structure | Active Protein Structure |
|---|---|
| SP-CTPx3-IGF1 | CTPx3 -IGF1 |
| SP-PP_{IGF1}-IGF1-CTPx4 | IGF1 -CTPx4 |
| SP-PP_{IGF1}-IGF1-CTPx3 | IGF1-CTPx3 |
| SP-CTP-IGF1-CTPx2 | CTP-IGF1-CTPx2 |
| SP-CTPx2-IGF1-CTPx4 | CTPx2-IGF1-CTPx4 |

In the above embodiments, the SP can be any signal peptide, the first pro-peptide PP can be IGF1 PP or any variant thereof; IGF-1 can be any active IGF-1 or variant thereof and CTP can be any CTP variant as described herein.

In another embodiment, CTP modified IGF-1 polypeptides are shown in Table 3.

TABLE 3

| CTP-Modified | | |
|---|---|---|
| Variant | Expressed Structure | Active Protein Structure |
| MOD-1301-1 | SP_{hGH}-CTPx3-IGF1 | CTPx3 -IGF1 |
| | (SEQ ID NO: 10) | (SEQ ID NO: 15) |
| MOD-1301-2 | SP_{IGF1}-PP_{IGF1}-IGF1-CTPx4 | IGF1 -CTPx4 |
| | (SEQ ID NO: 11) | (SEQ ID NO: 16) |
| MOD-1301-3 | SP_{IGF1}-PP_{IGF1}-IGF1-CTPx3 | IGF1-CTPx3 |
| | (SEQ ID NO: 12) | (SEQ ID NO: 17) |
| MOD-1301-4 | SP_{hGH}-CTP-IGF1-CTPx2 | CTP-IGF1-CTPx2 |
| | (SEQ ID NO: 13) | (SEQ ID NO: 18) |
| MOD-1301-5 | SP_{hGH}-CTPx2-IGF1-CTPx4 | CTPx2-IGF1-CTPx4 |
| | (SEQ ID NO: 14) | (SEQ ID NO: 19) |

The CTP-Modified Variants shown in Table 3 and named as MOD-1301-1-5 are specific constructs prepared according to the processes describe in the specification and in the examples.

In one embodiment, the amino acid sequence of the CTP-modified IGF-1, MOD-1301-1, comprises the following amino acid sequence:

(SEQ ID NO: 10)
MATGSRTSLLLAFGLLCLPWLQEGSASSSSKAPPPSLPSPSRLPGPSD
TPILPQSSSSKAPPPSLPSPSRLPGPSDTPILPQSSSSKAPPPSLPSP
SRLPGPSDTPILPQGPETLCGAELVDALQFVCGDRGFYFNKPTGYGSS
SRRAPQTGIVDECCFRSCDLRRLEMYCAPLKPAKSA.

In one embodiment, the amino acid sequence of the CTP-modified IGF-1, MOD-1301-2, comprises the following amino acid sequence:

(SEQ ID NO: 11)
MGKISSLPTQLFKCCFCDFLKVKMHTMSSSHLFYLALCLLTFTSSATA
GPETLCGAELVDALQFVCGDRGFYFNKPTGYGSSSRRAPQTGIVDECC
FRSCDLRRLEMYCAPLKPAKSASSSSKAPPPSLPSPSRLPGPSDTPIL
PQSSSSKAPPPSLPSPSRLPGPSDTPILPQSSSSKAPPPSLPSPSRLP
GPSDTPILPQSSSSKAPPPSLPSPSRLPGPSDTPILPQ.

In one embodiment, the amino acid sequence of the CTP-modified IGF-1, MOD-1301-3, comprises the following amino acid sequence:

(SEQ ID NO: 12)
MGKISSLPTQLFKCCFCDFLKVKMHTMSSSHLFYLALCLLTFTSSATAGP
ETLCGAELVDALQFVCGDRGFYFNKPTGYGSSSRRAPQTGIVDECCFRSC
DLRRLEMYCAPLKPAKSASSSSKAPPPSLPSPSRLPGPSDTPILPQSSSS
KAPPPSLPSPSRLPGPSDTPILPQSSSSKAPPPSLPSPSRLPGPSDTPIL
PQ.

In one embodiment, the amino acid sequence of the CTP-modified IGF-1, MOD-1301-4, comprises the following amino acid sequence:

(SEQ ID NO: 13)
MATGSRTSLLLAFGLLCLPWLQEGSASSSSKAPPPSLPSPSRLPGPSDTP
ILPQGPETLCGAELVDALQFVCGDRGFYFNKPTGYGSSSRRAPQTGIVDE
CCFRSCDLRRLEMYCAPLKPAKSASSSSKAPPPSLPSPSRLPGPSDTPIL
PQSSSSKAPPPSLPSPSRLPGPSDTPILPQ.

In one embodiment, the amino acid sequence of the CTP-modified IGF-1, MOD-1301-5, comprises the following amino acid sequence:

(SEQ ID NO: 14)
MATGSRTSLLLAFGLLCLPWLQEGSASSSSKAPPPSLPSPSRLPGPSDTP
ILPQSSSSKAPPPSLPSPSRLPGPSDTPILPQGPETLCGAELVDALQFVC
GDRGFYFNKPTGYGSSSRRAPQTGIVDECCFRSCDLRRLEMYCAPLKPAK
SASSSSKAPPPSLPSPSRLPGPSDTPILPQSSSSKAPPPSLPSPSRLPGP
SDTPILPQSSSSKAPPPSLPSPSRLPGPSDTPILPQSSSSKAPPPSLPSP
SRLPGPSDTPILPQ.

In another embodiment, the CTP-modified IGF-1 is a recombinant protein. In another embodiment, the CTP-modified IGF-1 is a recombinant glycoprotein. In another embodiment, the CTP-modified IGF-1 comprises a signal peptide. In another embodiment, a recombinant CTP-modified IGF-1 does not comprise a signal peptide. In one embodiment, the CTP-modified IGF-1 includes a signal peptide. In another embodiment, the CTP-modified IGF-1 does not include a signal peptide.

In one embodiment, following expression and prior to secretion, the signal peptides are cleaved from the precursor engineered CTP-modified IGF-1 resulting in the mature engineered CTP-modified IGF-1 lacking a signal peptide. In another embodiment, following expression and prior to secretion, both the signal peptide and the propeptide are cleaved from the precursor engineered CTP-modified IGF-1 resulting in the mature engineered CTP-modified IGF-1 lacking a signal peptide and lacking a propeptide.

In one embodiment, the amino acid sequence of the mature CTP-modified IGF-1, MOD-1301-1 without the signal peptide, comprises the following amino acid sequence:

(SEQ ID NO: 15)
SSSSKAPPPSLPSPSRLPGPSDTPILPQSSSSKAPPPSLPSPSRLPGPSD
TPILPQSSSSKAPPPSLPSPSRLPGPSDTPILPQGPETLCGAELVDALQF
VCGDRGFYFNKPTGYGSSSRRAPQTGIVDECCFRSCDLRRLEMYCAPLKP
AKSA.

In one embodiment, the amino acid sequence of the mature CTP-modified IGF-1, MOD-1301-2 without both the signal peptide and the first propeptide, comprises the following amino acid sequence:

(SEQ ID NO: 16)
GPETLCGAELVDALQFVCGDRGFYFNKPTGYGSSSRRAPQTGIVDECCFR
SCDLRRLEMYCAPLKPAKSASSSSKAPPPSLPSPSRLPGPSDTPILPQSS
SSKAPPPSLPSPSRLPGPSDTPILPQSSSSKAPPPSLPSPSRLPGPSDTP
ILPQSSSSKAPPPSLPSPSRLPGPSDTPILPQ.

In one embodiment, the amino acid sequence of the mature CTP-modified IGF-1, MOD-1301-3 without both the signal peptide and the first propeptide, comprises the following amino acid sequence:

(SEQ ID NO: 17)
GPETLCGAELVDALQFVCGDRGFYFNKPTGYGSSSRRAPQTGIVDECCIR
SCDLRRLEMYCAPLKPAKSASSSSKAPPPSLPSPSRLPGPSDTPILPQSS
SSKAPPPSLPSPSRLPGPSDTPILPQSSSSKAPPPSLPSPSRLPGPSDTP
ILPQ.

In one embodiment, the amino acid sequence of the mature CTP-modified IGF-1, MOD-1301-4 without the signal peptide, comprises the following amino acid sequence:

(SEQ ID NO: 18)
SSSSKAPPPSLPSPSRLPGPSDTPILPQGPETLCGAELVDALQFVCGDRG
FYFNKPTGYGSSSRRAPQTGIVDECCFRSCDLRRLEMYCAPLKPAKSASS
SSKAPPPSLPSPSRLPGPSDTPILPQSSSSKAPPPSLPSPSRLPGPSDTP
ILPQ.

In one embodiment, the amino acid sequence of the mature CTP-modified IGF-1, MOD-1301-5 without the signal peptide, comprises the following amino acid sequence:

(SEQ ID NO: 19)
SSSSKAPPPSLPSPSRLPGPSDTPILPQSSSSKAPPPSLPSPSRLPGPSD
TPILPQGPETLCGAELVDALQFVCGDRGFYFNKPTGYGSSSRRAPQTGIV
DECCFRSCDLRRLEMYCAPLKPAKSASSSSKAPPPSLPSPSRLPGPSDTP
ILPQSSSSKAPPPSLPSPSRLPGPSDTPILPQSSSSKAPPPSLPSPSRLP
GPSDTPILPQSSSSKAPPPSLPSPSRLPGPSDTPILPQ.

In one embodiment, the CTP sequence of the present invention comprises at least one glycosylation site. In one embodiment, the CTP sequence of the present invention comprises 2 glycosylation sites. In one embodiment, the CTP sequence of the present invention comprises 3 glycosylation sites. In one embodiment, the CTP sequence of the present invention comprises 4 glycosylation sites. In one embodiment, the CTP sequence of the present invention comprises 5 glycosylation sites. In one embodiment, the CTP sequence of the present invention comprises 6 glycosylation sites. In one embodiment, the CTP sequence of the present invention comprises 7 glycosylation sites. In one embodiment, the CTP sequence of the present invention comprises 8 glycosylation sites.

In one embodiment, the CTP-modified IGF-1 comprises 1 to 8 O-linked glycosylation sites occurring on any amino acid residues present at each attached CTP. In another embodiment, the CTP-modified IGF-1 comprises 4 to 6 O-linked glycosylation sites occurring on any amino acid residues present at each attached CTP. In another embodiment, each CTP in the CTP-modified IGF-1 contains 4, 5, or 6 O-linked glycans.

In one embodiment, the CTP sequence of the CTP-modified IGF-1 is glycosylated at all the serine residues present on the CTP sequence.

In one embodiment, one or more of the chorionic gonadotropin CTP amino acid sequences is fully glycosylated. In another embodiment, one or more of the chorionic gonadotropin CTP amino acid sequences is partially glycosylated. In one embodiment, partially glycosylated indicates that one of the CTP glycosylation sites is glycosylated. In another embodiment, two of the CTP glycosylation sites are glycosylated. In another embodiment, three of the CTP glycosylation sites are glycosylated. In another embodiment, 4 to 6 of the CTP glycosylation sites are glycosylated. In another embodiment, 7 to 8 of the CTP glycosylation sites are glycosylated.

In one embodiment, the CTP-modified IGF-1 or IGF-1 variants disclosed herein bind to an Insulin receptor with an average EC₅₀ value of between 100 nM and 400 nM. In another embodiment, the CTP-modified IGF-1 or IGF-1 variants disclosed herein bind to an Insulin receptor with an average EC₅₀ value of approximately 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, or 400 nM.

In one embodiment, the CTP-modified IGF-1 or IGF-1 variant disclosed herein bind to an IGF-1 receptor with an average EC₅₀ value of between 1 nM and 3 nM. In another embodiment, the CTP-modified IGF-1 or IGF-1 variant disclosed herein bind to an IGF-1 receptor with an average EC₅₀ value of approximately 1.0 nM, 1.1 nM, 1.2 nM, 1.3 nM, 1.4 nM, 1.5 nM, 1.6 nM, 1.7 nM, 1.8 nM, 1.9 nM, 2.0 nM, 2.1 nM, 2.2 nM, 2.3 nM, 2.4 nM, 2.5 nM, 2.6 nM, 2.7 nM, 2.8 nM, 2.9 nM, or 3.0 nM.

In one embodiment, the CTP-modified IGF-1 or IGF-1 disclosed herein bind to an Insulin receptor and bind to an IGF-1 receptor with an average EC₅₀ value (EC₅₀ Insulin receptor/EC₅₀ IGF-1 receptor) that is present in a ratio of between 30 to 400. In another embodiment, the CTP-modified IGF-1 or IGF-1 disclosed herein bind to an Insulin receptor and bind to an IGF-1 receptor with an average EC₅₀ value (EC₅₀ Insulin receptor/EC₅₀ IGF-1 receptor) that is present in a ratio of approximately 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400. In another embodiment, the CTP-modified IGF-1 or IGF-1 disclosed herein bind to an Insulin receptor and bind to an IGF-1 receptor with an average EC₅₀ value (EC₅₀ Insulin receptor/EC₅₀ IGF-1 receptor) that is present in a ratio of approximately 155, 156, 157, 158, 159, 160, 161, 162, 163, 164, 165, 166, or 167. In another embodiment, the CTP-modified IGF-1 or IGF-1 disclosed herein bind to an Insulin receptor and bind to an IGF-1 receptor with an average EC₅₀ value (EC₅₀ Insulin receptor/EC₅₀ IGF-1 receptor) that is present in a ratio of approximately 115, 116, 117, 118, 119, 120, 121, 122, 123, 124, 125, 126, 127, 128, 129, 130.

### Polynucleotides

In one embodiment, the polynucleotide encoding a long acting IGF-1 comprises a single cgCTP attached to the amino terminus of said IGF-1. In another embodiment, the polynucleotide encoding a long acting IGF-1 comprises two cgCTPs at the amino terminus of said IGF-1. In another embodiment, the polynucleotide encoding a long acting IGF-1 comprises three cgCTPs at the amino terminus of said IGF-1. In another embodiment, the polynucleotide encoding a long acting IGF-1 comprises four cgCTPs at the amino terminus of said IGF-1. In another embodiment, the polynucleotide encoding a long acting IGF-1 comprises five cgCTPs at the amino terminus of said IGF-1. In another embodiment, the polynucleotide encoding a long acting IGF-1 comprises one to five cgCTPs at the amino terminus of said IGF-1 and no cgCTPs attached to the carboxy terminus.

In one embodiment, the polynucleotide encoding a long acting IGF-1 comprises a single cgCTP at the carboxy terminus of said IGF-1. In another embodiment, the polynucleotide encoding a long acting IGF-1 comprises two cgCTPs at the carboxy terminus of said IGF-1. In another embodiment, the polynucleotide encoding a long acting IGF-1 comprises three cgCTPs at the carboxy terminus of said IGF-1. In another embodiment, the polynucleotide encoding a long acting IGF-1 comprises four cgCTPs at the carboxy terminus of said IGF-1. In another embodiment, the polynucleotide encoding a long acting IGF-1 comprises five cgCTPs at the carboxy terminus of said IGF-1. In another embodiment, the polynucleotide encoding a long acting IGF-1 comprises one to five cgCTPs at the carboxy terminus of said IGF-1 and no cgCTPs attached to the amino terminus.

In one embodiment, the polynucleotide encoding a long acting IGF-1 comprises a single cgCTP attached to the amino terminus and a single cgCTP at the carboxy terminus. In another embodiment, the polynucleotide encoding a long acting IGF-1 comprises a single cgCTP at the amino terminus and two cgCTPs at the carboxy terminus. In another the polynucleotide encoding a long acting IGF-1 comprises a single cgCTP at the amino terminus and three cgCTPs at the carboxy terminus. In another embodiment, the polynucleotide encoding a long acting IGF-1 comprises a single cgCTP at the amino terminus and four cgCTPs at the carboxy terminus. In another embodiment, the polynucleotide encoding a long acting IGF-1 comprises a single cgCTP at the amino terminus and five cgCTPs at the carboxy terminus.

In one embodiment, the polynucleotide encoding a long acting IGF-1 comprises a single cgCTP attached to the carboxy terminus and a single cgCTP at the amino terminus. In another embodiment, the polynucleotide encoding a long acting IGF-1 comprises a single cgCTP at the carboxy terminus and two cgCTPs at the amino terminus. In another embodiment, the polynucleotide encoding a long acting IGF-1 comprises a single cgCTP at the carboxy terminus and three cgCTPs at the amino terminus. In another embodiment, the polynucleotide encoding a long acting IGF-1 comprises a single cgCTP at the carboxy terminus and four cgCTPs at the amino terminus. In another embodiment, the polynucleotide encoding a long acting IGF-1 comprises a single cgCTP at the carboxy terminus and five cgCTPs at the amino terminus.

In another embodiment, provided herein is an expression vector comprising a polynucleotide comprising a CTP-modified IGF-1.

In another embodiment, the CTP-modified IGF-1 polypeptides of the present invention are synthesized using a polynucleotide molecule encoding a polypeptide of the present invention. In another embodiment, the polynucleotide molecule encoding the CTP-modified IGF-1 of the present invention is ligated into an expression vector, comprising a transcriptional control of a cis-regulatory sequence (e.g., promoter sequence). In another embodiment, the cis-regulatory sequence is suitable for directing constitutive expression of a CTP-modified IGF-1 of the present invention. In another embodiment, the cis-regulatory sequence is suitable for directing tissue-specific expression of a CTP-modified IGF-1 of the present invention. In another embodiment, the cis-regulatory sequence is suitable for directing inducible expression of the CTP-modified IGF-1 polypeptides of the present invention.

In one embodiment, the nucleic acid sequence encoding the CTP-modified IGF-1, MOD-1301-1, comprises the following nucleic acid sequence:

(SEQ ID NO: 20)
gcggccgccatggccaccgggagccggacatctctgctgctggctttcgg
tctgctgtgcctgccatggctgcaggagggcagtgcttccagctctagta
aggcaccccctccatcactgccttccccttctagactgcctggaccatct
gacaccccaatcctgcctcagtcatccagctctaaagctccccctccatc
tctgccttctccaagtcgtctgcccgggcctagtgatacaccaattctgc
cccagagttcatccagcaaggcaccccctccaagcctgccatcaccatcc
aggctgccaggcccatctgacactcctatcctgccacagggacctgagac
cctgtgcggagcagaactggtggacgccctgcagttcgtctgtggggata
gaggtttctactttaacaaacccacaggctatggatctagttcaaggcgg
gcacctcagactggcattgtggacgagtgctgttttaggtcctgcgatct
gagacgcctggaaatgtactgtgcccctctgaagccagccaaatccgcct
gataagctttga

In one embodiment, the nucleic acid sequence encoding the CTP-modified IGF-1, MOD-1301-2, comprises the following nucleic acid sequence:

(SEQ ID NO: 21)
gcggccgccatgggcaagatctccagcctgcctacccagctgttcaaatg
ctgtttctgcgactttctgaaggtgaaaatgcacacaatgtctagttcac
acctgttctacctggccctgtgcctgctgacctttacatccagcgccact
gctggaccagagaccctgtgcggagctgaactggtggacgcactgcagac
gtctgtggggataggggtttctactttaacaagccaacaggctatggatc
tagttcaaggcgggcccctcagactgggattgtcgacgagtgctgttttc
ggagctgcgatctgagacgcctggaaatgtattgtgcccctctgaagcca
gcaaaatcagcctccagctctagtaaggctccccctccaagtctgcctag
cccttctagactgcctggaccatctgacactccaatcctgcctcagtcat
ccagctctaaagcaccccctccaagcctgcctagtccatcacgtctgccc
ggtccttctgataccccaattctgccccagagttcatccagcaaggcccc
tcccccatccctgccttctcctagcaggctgccaggcccatctgacacac
ctatcctgccacagtctagttcatccaaagctccccctccatctctgccc
tctcctagtagactgccaggaccctccgatacccccattctgcctcagtg
ataagctttga.

In one embodiment, the nucleic acid sequence encoding the CTP-modified IGF-1, MOD-1301-3, comprises the following nucleic acid sequence:

(SEQ ID NO: 22)
gcggccgccatgggcaagatctcttcactgcccacccagttgttcaagtg
ctgtttctgcgactttctgaaggtgaagatgcacaccatgagtagctcac
acctgttttatctggccctctgtctgctcaccttcacttctagtgccact
gccggaccagaaaccctctgcggcgccgaactggtggacgcattgcagtt
cgtgtgcggagacaggggtttctactttaacaagccaacaggttacggct
cctctagcagacgggctccccagaccggcatcgttgatgagtgctgtttt
aggtcctgtgacctcaggcgtctggagatgtattgcgctcccctgaaacc
agccaagtctgcaagctcatcaccaaggcacctccaccttctctgccaag
cccctctaggttgccaggcccttccgatacccccattttgcctcagtcat
ccagcagtaaggcaccacccccttccctgcctagcccttcaaggctgcca
ggccctagcgataccccaattctgccacagagctcaagctccaaagcccc
acctccctcactgccatccccttctcggctgccaggcccatccgataccc
ctatcttgccacagtgataagctttga.

In one embodiment, the nucleic acid sequence encoding the CTP-modified IGF-1, MOD-1301-4, comprises the following nucleic acid sequence:

(SEQ ID NO: 23)
gcggccgccatggctaccggtagtaggactagcctgctcaggcatttggt
ctgctctgtctgccttggttgcaggagggcagtgcctccagctcctctaa
agctcctccaccctctttgccaagcccctctagattgcctggtccatccg
atactccaattctgcctcagggccctgagactttgtgcggcgctgaactg
gtggacgcactccagttcgtctgcggagacagaggcttctacttcaacaa
acctactgggtatggttcttccagtcgtagggcaccacagacaggtatcg
tggatgagtgttgcttcaggtcatgtgacctcaggcgtctggagatgtac
tgtgcaccactgaagcctgcaaaatccgcctcaagctccagtaaggctcc
acctccttcattgccaagcccttctcgtctgcccggtccaagcgacaccc
caattctgccccagtcatcttccagcaaagccccacctccaagtctgccc
agcccaagtcgactgcctggaccctctgatacccccatcctgccacagtg
ataagctttga.

In one embodiment, the nucleic acid sequence encoding the CTP-modified IGF-1, MOD-1301-5, comprises the following nucleic acid sequence:

(SEQ ID NO: 24)
gcggccgccatggcaacaggtagtaggacttctttgctgctcgcctttgg
actgctgtgcctcccttggctgcaggagggctcagctagcagcagttcca
aggctcctcccccatctctgccttcacccagcaggttgcccgggccatca
gatactccaatcctcccccagtcttccagtagcaaagccccacctccctc
cctgccttcaccatccaggttgcctggtccaagcgatacacctatcctgc
cacagggacctgagacactctgtggtgcagagctggtggatgcattgcag
tttgtttgcggcgacagagggttctacttcaacaagcctactggctatgg
ttctagctccagaagagcaccacagaccggaatcgtggatgaatgctgct
tccgttcctgcgacttgcgcagactggagatgtattgtgccccactcaaa
cctgctaagtccgccagttctagctccaaagctcctccaccctcactgcc
cagcccatcaaggctcccaggaccctcagatacccccattttgcctcagt
ctagctccagcaaggcacctccaccctctttgccctctccaagcagattg
ccaggtcctagtgacactcccatcctgcctcagtcaagctccagtaaagc
ccctccacctagcctcccatctcccagcagactgccaggtcctagcgata
cacccatcttgccccagtcaagtagctccaaagctccaccccctagcctc
ccttcaccctctaggttgcctggcccatcagatacaccaattctcccaca
gtgataagctttga

In one embodiment, tissue-specific promoters suitable for use with the present invention include sequences which are functional in one or more specific cell populations. Examples include, but are not limited to, promoters such as albumin that is liver-specific [Pinkert et al., (1987) Genes Dev. 1:268-277], lymphoid-specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). Inducible promoters suitable for use with the present invention include, for example, the tetracycline-inducible promoter (Srour, M. A., et al., 2003. Thromb. Haemost. 90: 398-405).

In one embodiment, the phrase “a polynucleotide molecule” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In another embodiment, provided herein is a composition comprising the polypeptide, polynucleotide, expression vector, or a combination thereof.

### Therapeutic Compositions

In one embodiment, a “pharmaceutical composition” or a “pharmaceutical formulation” refers to a preparation of one or more of the active ingredients described herein with other chemical components such as physiologically suitable carriers and excipients. The purpose of a pharmaceutical composition or a “pharmaceutical formulation” is to facilitate administration of a compound to an organism. In certain embodiments, a “pharmaceutical composition” or a “pharmaceutical formulation” provides the pharmaceutical dosage form of a drug. “Pharmaceutical compositions” or “pharmaceutical formulations” in certain embodiments include slow release technologies, transdermal patches, or any known dosage form in the art.

As used herein, “alleviating a symptom of IGFD” refers to achieving a therapeutic benefit for a symptom associated with IGF-1 deficiency. Symptoms of IGFD patients include, but are not limited to, decreased growth rate and height, increased blood pressure, decreased cardiac performance, cardiac disease, renal disease, neurological disease, impaired exercise performance, decreased muscle mass, decreased bone density, obesity and abnormalities of carbohydrate and lipid metabolism. Thus, alleviating symptoms of IGFD results in increased growth rates and height, bone density, bone structure, improved renal and cardiac function, and improved glucose control and body composition.

As used herein, “treatment” or “treating” refers to inhibiting the progression of a disease or disorder, e.g., short stature or IGFD, or delaying the onset of a disease or disorder, e.g., short stature or IGFD, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect may be prophylactic in terms of completely or partially preventing a disease or condition, or a symptom thereof and/or may be therapeutic in terms of a partial or complete cure for a disease or disorder and/or adverse affect attributable to the disease or disorder. “Treatment,” as used herein, covers any treatment of a disease or disorder in a mammal, such as a human, and includes: decreasing the risk of death due to the disease; preventing the disease of disorder from occurring in a subject which may be predisposed to the disease but has not yet been diagnosed as having it; inhibiting the disease or disorder, i.e., arresting its development (e.g., reducing the rate of disease progression); and relieving the disease, i.e., causing regression of the disease. Therapeutic benefits of the present invention include, but are not necessarily limited to, reduction of risk of onset or severity of disease or conditions associated with short stature or IGFD.

As used herein, a “therapeutically effective amount” refers to that amount of the compound sufficient to treat or manage a disease or disorder, e.g., short stature or IGFD. A therapeutically effective amount may refer to the amount of a compound that provides a therapeutic benefit in the treatment or management of a disease or disorder. Further, a therapeutically effective amount with respect to a compound of the invention means that amount of compound alone, or in combination with other therapies, that provides a therapeutic benefit in the treatment or management of a disease or disorder. The term can encompass an amount that improves overall therapy, reduces or avoids unwanted effects, or enhances the therapeutic efficacy of or synergies with another therapeutic agent.

In another embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

It is to be understood that the compositions, formulations and methods of the present invention comprising the elements or steps as described herein may, in another embodiment, consist of those elements or steps, or in another embodiment, consist essentially of those elements or steps. In some embodiments, the term “comprise” refers to the inclusion of the indicated active agent, such as the CTP-modified IGF-1, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In another embodiment, the pharmaceutical compositions and pharmaceutical formulations are administered by intravenous, subcutaneous, intra-arterial, or intramuscular injection of a liquid preparation. In some embodiments, liquid formulations include solutions, suspensions, dispersions, emulsions, oils and the like. In one embodiment, the pharmaceutical compositions and pharmaceutical formulations are administered intravenously, and are thus formulated in a form suitable for intravenous administration. In another embodiment, the pharmaceutical compositions and pharmaceutical formulations are administered intra-arterially, and are thus formulated in a form suitable for intra-arterial administration. In another embodiment, the pharmaceutical compositions and pharmaceutical formulations are administered intramuscularly, and are thus formulated in a form suitable for intramuscular administration.

In another embodiment, the pharmaceutical compositions and pharmaceutical formulations are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, the pharmaceutical composition disclosed herein comprises CTP-modified IGF-1.

In one embodiment, pharmaceutical compositions and pharmaceutical formulations of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions and pharmaceutical formulations for use in accordance with the present invention are formulated in a conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

### Manufacturing

In one aspect, the invention provided herein relates to a method of manufacturing a human chorionic gonadotropin peptide (CTP)-modified human IGF-1 polypeptide, the method comprising the steps of: a. stably transfecting a predetermined number of cells with an expression vector comprising a coding portion encoding said CTP-modified IGF-1, wherein said transfected cell expresses said CTP-modified IGF-1; b. obtaining cell clones that overexpress said CTP-modified IGF-1; c. expanding said clones in solution to a predetermined scale; d. harvesting said solution containing said clones; e. filtering said solution containing said clones to obtain a clarified harvest solution; and, f. purifying said clarified harvest solution to obtain a purified protein solution having a desired concentration of a CTP-modified IGF-1, thereby manufacturing a human chorionic gonadotropin peptide (CTP)-modified IGF-1 polypeptide.

In one embodiment, a preliminary purification process comprises sequentially performing steps comprising passing said clarified harvest through affinity column, an anion exchange column; the anion exchange eluate undergoes an ultrafiltration/diafiltration step

In another embodiment, the CTP-modified IGF-1 of the present invention are synthesized using a polynucleotide encoding a polypeptide of the present invention. In another embodiment, the polynucleotide encoding the CTP-modified IGF-1 of the present invention is ligated into an expression vector, comprising a transcriptional control of a cis-regulatory sequence (e.g., promoter sequence). In another embodiment, the cis-regulatory sequence is suitable for directing constitutive expression of the CTP-modified IGF-1 of the present invention. In another embodiment, the cis-regulatory sequence is suitable for directing tissue specific expression of the CTP-modified IGF-1 of the present invention. In another embodiment, the cis-regulatory sequence is suitable for directing inducible expression of the CTP-modified IGF-1 of the present invention.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

Various methods, in some embodiments, can be used to introduce the expression vector of the present invention into the host cell system. In some embodiments, such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, transformed cells are cultured under effective conditions, which allow for the expression of high amounts of recombinant polypeptide. In another embodiment, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In another embodiment, a medium typically includes an aqueous solution having assimilable carbon, nitrogen and phosphate sources, and appropriate salts, minerals, metals and other nutrients, such as vitamins. In another embodiment, cells of the present invention can be cultured in conventional fermentation bioreactors, shake flasks, test tubes, microtiter dishes and petri plates. In another embodiment, culturing is carried out at a temperature, pH and oxygen content appropriate for a recombinant cell. In another embodiment, culturing conditions are within the expertise of one of ordinary skill in the art.

In another embodiment, depending on the vector and host system used for production, resultant IGF 1—of the present invention either remain within the recombinant cell, secreted into the fermentation medium, secreted into a space between two cellular membranes, such as the periplasmic space in E. coli; or retained on the outer surface of a cell or viral membrane.

In one embodiment, following a predetermined time in culture, recovery of the recombinant polypeptide is effected.

In one embodiment, the phrase “recovering the recombinant polypeptide” used herein refers to collecting the whole fermentation medium containing the polypeptide and need not imply additional steps of separation or purification.

In one embodiment, CTP-modified IGF-1 of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, to facilitate recovery, the expressed coding sequence can be engineered to encode the polypeptide of the present invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In one embodiment, the polypeptide of the present invention is retrieved in “substantially pure” form.

In one embodiment, the phrase “substantially pure” refers to a purity that allows for the effective use of the protein in the applications described herein

In one embodiment, the polypeptide of the present invention can also be synthesized using in vitro expression systems. In one embodiment, in vitro synthesis methods are well known in the art and the components of the system are commercially available.

In another embodiment, the recombinant polypeptides are synthesized and purified; their therapeutic efficacy can be assayed either in vivo or in vitro. In one embodiment, the binding activities of the recombinant IGF-1 modified by CTPs of the present invention can be ascertained using various assays.

Other features and advantages will become apparent from the following detailed description, examples, and figures. It should be understood, however, that the detailed description and the specific examples while indicating preferred embodiments of the disclosure are given by way of illustration only, since various changes and modification within the spirit and scope of the disclosure will become apparent to those skilled in the art from this

### Methods of Use

In one embodiment, the present invention provides a method of treating IGF-1 related disease or disorder in a subject comprising the step of administering to said subject a polypeptide of a CTP-modified IGF-1 or IGF-1 variant as described herein. In another aspect, administering is via a subcutaneous or intramuscular route. In another aspect, administering is via the intravenous route.

As used herein, “insulin-like growth factor-1 deficiency”, “IGF-1 deficiency”, or “IGFD” refer to a condition associated with the following characteristics, a height of at least about 2 standard deviations (SD) below the normal mean level for the corresponding age and gender, a blood level of IGF-1 that is at least 1 SD below normal mean levels. In general, IGFD can be due to a resistance to GH action or as a result of GH deficiency (GHD). IGFD that is due to resistance to GH action is termed primary IGFD, while IGFD resulting from GHD is termed secondary IGFD. Primary IGFD is distinguished from secondary IGFD in that primary IGFD is associated with at least normal GH blood levels, while secondary IGFD is associated with low blood levels of GH.

Thus, primary IGFD refers to a condition associated with the following characteristics, a height of at least about 2 standard deviations (SD) below the normal mean for the corresponding age and gender, a blood level of IGF-1 that is below normal mean levels, and a mean or maximum stimulated blood level of growth hormone (GH) that is at least normal (e.g., normal GH blood levels or greater than normal GH blood levels). Generally, the normal GH blood levels correspond to levels above 7. GHBP levels are generally within the normal range.

In one aspect, blood glucose levels are measured by methods known in the art such as a valid glucometer.

Pediatric primary IGFD refers to pediatric patients with IGFD, while Adult primary IGFD refers to adult patients with IGFD. Adult primary IGFD, is similar to pediatric primary IGFD and is associated with a height of at least 2 SD below the normal mean for the corresponding age and gender, a blood level of IGF-1 that is at least 2 SD below the normal mean for the corresponding age and gender, and normal growth hormone levels. Adult primary IGFD patients have increased blood pressure, decreased cardiac performance, cardiac disease, renal disease impaired exercise performance, decreased muscle mass, decreased bone density, obesity and abnormalities of carbohydrate and lipid metabolism. Pediatric patients with primary IGFD are capable of having their height or growth rate increased, while adult patients are no longer capable of achieving a greater height.

In yet other aspects the invention features a method for treating a subject having a primary insulin-like growth factor-1 deficiency (IGFD) comprising administering to a human subject having primary insulin-like growth factor-1 deficiency (IGFD) an effective amount of CTP-modified IGF-1, wherein the subject is characterized as follows: a) at the time of treatment or prior to initial treatment with IGF-1, has or had a height at least about 2 standard deviations (SD) below a normal mean for a corresponding age and gender, b) the time of treatment or prior to initial treatment with IGF-1, has or had a blood level of IGF-1 at least about −1 SD below normal mean levels, and c) at a blood level of growth hormone (GH) which is at least normal, wherein the subject does not have Laron syndrome or partial growth hormone insensitivity syndrome, and wherein said administering provides for treatment of IGFD in the subject.

Thus in one embodiment, methods of the present invention are for treating the diseases, disorders, and symptoms associated with IGF deficiency, as described herein.

In one embodiment, disclosed herein is a method of treating a subject having growth failure with severe primary IGF-1 deficiency by administering an effective amount of CTP-modified IGF-1 variants disclosed throughout the application. In another embodiment, the subject is an adult. In another embodiment, the subject is an IGF-1 deficient adult. In one embodiment, disclosed herein is a method of treating an adult subject having growth failure with severe primary IGF-1 deficiency by administering an effective amount of CTP-modified IGF-1 variants disclosed throughout the application. In another embodiment, the subject is a child. In another embodiment, the subject is an IGF-1 deficient child. In one embodiment, disclosed herein is a method of treating a child subject having growth failure with severe primary IGF-1 deficiency by administering an effective amount of CTP-modified IGF-1 variants disclosed throughout the application.

In one embodiment, disclosed herein is a method of treating a subject with growth hormone (GH) gene deletion who have developed neutralizing antibodies to GH by administering an effective amount of CTP-modified IGF-1 variants disclosed throughout the application. In another embodiment, the subject is an adult. In another embodiment, the subject is an IGF-1 deficient adult. In one embodiment, disclosed herein is a method of treating an adult subject with growth hormone (GH) gene deletion who have developed neutralizing antibodies to GH by administering an effective amount of CTP-modified IGF-1 variants disclosed throughout the application. In another embodiment, the subject is a child. In another embodiment, the subject is an IGF-1 deficient child. In one embodiment, disclosed herein is a method of treating a child subject with growth hormone (GH) gene deletion who have developed neutralizing antibodies to GH by administering an effective amount of CTP-modified IGF-1 variants disclosed throughout the application.

In one embodiment, disclosed herein is a method of treating a subject having severe primary IGF-1 deficiency (Primary IGFD) by administering an effective amount of CTP-modified IGF-1 variants disclosed throughout the application. In another embodiment, the subject is an adult. In another embodiment, the subject is an IGF-1 deficient adult. In one embodiment, disclosed herein is a method of treating an adult subject having severe primary IGF-1 deficiency (Primary IGFD) by administering an effective amount of CTP-modified IGF-1 variants disclosed throughout the application. In another embodiment, the subject is a child. In another embodiment, the subject is an IGF-1 deficient child. In one embodiment, disclosed herein is a method of treating a child subject having severe primary IGF-1 deficiency (Primary IGFD) by administering an effective amount of CTP-modified IGF-1 variants disclosed throughout the application.

In one embodiment, disclosed is a method of treating a patient having an IGF-1 related disease or disorder comprising administering a pharmaceutically effective amount of any of the CTP-modified IGF-1 described herein, wherein said CTP modified IGF-1 has reduced hypoglycemic side effects relative to an equal molar dose of an IGF-1. In one embodiment, disclosed is a method of treating a patient having an IGF-1 related disease or disorder comprising administering a pharmaceutically effective amount of any of the CTP-modified IGF-1 described herein, wherein said CTP modified IGF-1 has reduced hypoglycemic side effects relative to an equal molar dose of the IGF-1 antagonist, Increlex. In one embodiment, the present invention relates to a method of treating a subject in need of treatment thereof with a CTP modified IGF-1 polypeptide or variant thereof comprising administering to said subject a therapeutically effective dose to treat an IGF-1 related disease, disorder or condition and wherein hypoglycemic effects are reduced relative to treatment with an equivalent molar dose of the non-CTP modified IGF-1 polypeptide and wherein said hypoglycemic effects are measured as a change in glucose blood levels (% from basal) over a 0-1, 0-2, 0-3, 0-4, 0-5 or 0-6 hour time period. In one embodiment, disclosed is a method of treating a patient having an IGF-1 related disease or disorder comprising administering a pharmaceutically effective amount of any of the CTP-modified IGF-1 described herein, wherein said CTP modified IGF-1 has reduced hypoglycemic side effects relative to an equal molar dose of the IGF-1 antagonist, Increlex.

In one embodiment, the potency of the CTP-modified IGF-1 or IGF-1 variant to activate the insulin receptor is much lower compared to endogenous IGF-1 or the IGF-1 antagonist, Increlex.

In one embodiment, the CTP-modified IGF-1 or IGF-1 variants described herein are co-administered to a patient in need thereof with hGH or an insulin-like growth factor binding protein (“IGFBP”). In another embodiment, the IGFBP being co-administered is IGFBP-3. In another embodiment, the IGFBP-3 is recombinant human IGFBP-3 (rhIGFBP-3). In another embodiment, the IGFBP being co-administered is IGFBP-3 or an analog thereof.

In one embodiment, the CTP-modified IGF-1 or IGF-1 variants described herein are co-administered to a patient in need thereof with an estrogen hormone. In another embodiment, the estrogen hormone co-administered with the CTP-modified IGF-1 or IGF-1 variants described throughout are transdermal formulations, such as a patch, spray, or topical emulsion. In another embodiment, the estrogen hormone co-administered with the CTP-modified IGF-1 or IGF-1 variants described throughout are oral formulations. In another embodiment, the estrogen hormone co-administered with the CTP-modified IGF-1 or IGF-1 variants described throughout are subdural, subcutaneous, or intravenous formulations.

In one embodiment, the CTP-modified IGF-1 or IGF-1 variants described herein are co-administered to a patient in need thereof with CTP-modified human growth hormone (hGH) polypeptide. In one embodiment, the CTP-modified IGF-1 or IGF-1 variants described herein are co-administered to a patient in need thereof with CTP-modified versions of human growth hormone (hGH). In one embodiment, the CTP-modified hGH polypeptide disclosed herein comprises carboxy terminal peptide of human Chorionic Gonadotropin (CTP).

In another embodiment, the CTP-modified hGH disclosed herein is a polypeptide consisting of a growth hormone, a single human chorionic gonadotropin carboxy terminal peptide (CTP) attached to the amino terminus of the human growth hormone (hGH), and two human chorionic gonadotropin carboxy terminal peptides (CTPs) attached to the carboxy terminus of the GH, wherein said polypeptide lacks a signal peptide, and said CTP-modified hGH polypeptide comprises the amino acid sequence as set forth in SEQ ID NO: 26. In another embodiment, disclosed herein is a CTP-modified hGH polypeptide consisting of a GH, a single CTP attached to the amino terminus of the GH, two CTPs attached to the carboxy terminus of the GH, and a signal peptide attached to the amino terminus of the amino terminal CTP, said polypeptide comprising the amino acid sequence as set forth in SEQ ID NO: 27. A skilled artisan would appreciate that a mature secreted polypeptide lacks a signal peptide.

In another embodiment, disclosed herein is a polypeptide consisting of a GH, a single CTP attached to the amino terminus of the GH, two CTPs attached to the carboxy terminus of the GH, and a signal peptide attached to the amino terminus of the N-terminal CTP, said polypeptide having the amino acid sequence set forth in SEQ ID NO: 26. A skilled artisan would appreciate that a mature, secreted polypeptide may lack a signal peptide. Thus, in yet another embodiment, disclosed herein is a polypeptide consisting of a GH, a single CTP attached to the amino terminus of the GH, two CTPs attached to the carboxy terminus of the GH, and no signal peptide, said polypeptide having the amino acid sequence set forth in SEQ ID NO: 27.

In one embodiment, a CTP-modified hGH precursor polypeptide disclosed herein is set forth in SEQ ID NO: 27:

(SEQ ID NO: 27)
MATGSRTSLLLAFGLLCLPWLQEGSASSSSKAPPPSLPSPSRLPGPSDTP
ILPQFPTIPLSRLFDNAMLRAHRLHQLAFDTYQEFEEAYIPKEQKYSFLQ
NPQTSLCFSESIPTPSNREETQQKSNLELLRISLLLIQSWLEPVQFLRSV
FANSLVYGASDSNVYDLLKDLEEGIQTLMGRLEDGSPRTGQIFKQTYSKF
DTNSHNDDALLKNYGLLYCFRKDMDKVETFLRIVQCRSVEGSCGFSSSSK
APPPSLPSPSRLPGPSDTPILPQSSSSKAPPPSLPSPSRLPGPSDTPILP
Q.

In another embodiment, the polypeptides disclosed herein provide a mature CTP-modified hGH lacking a signal peptide as set forth in SEQ ID NO: 26.

In one embodiment, following expression and secretion of a CTP-modified hGH polypeptide, disclosed herein, the signal peptide is cleaved from the precursor protein resulting in a mature protein. For example, in SEQ ID NO: 27, amino acids 1-26, MATGSRTSLLLAFGLLCLPWLQEGSA represent the signal peptide of the CTP-modified hGH polypeptide, and amino acids SSSSKAPPPSLPSPSRLPGPSDTPILPQFPTIPLSRLFDNAMLRAHRLHQLAFDTYQEFEEA YIPKEQKYSFLQNPQTSLCFSESIPTPSNREETQQKSNLELLRISLLLIQSWLEPVQFLRSVF ANSLVYGASDSNVYDLLKDLEEGIQTLMGRLEDGSPRTGQIFKQTYSKFDTNSHNDDA LLKNYGLLYCFRKDMDKVETFLRIVQCRSVEGSCGFSSSSKAPPPSLPSPSRLPGPSDTPI LPQSSSSKAPPPSLPSPSRLPGPSDTPILPQ (SEQ ID NO: 26) represent the mature engineered CTP-modified hGH polypeptide lacking the signal peptide.

In another embodiment, the CTP-modified hGH has enhanced in vivo biological activity compared with the same hGH without CTPs.

In another embodiment, the methods disclosed herein comprise use of a nucleic acid sequence encoding a CTP-modified hGH polypeptide disclosed herein. In one embodiment, the methods disclosed herein comprise use of the nucleic acid set forth in SEQ ID NO: 28 encoding an hGH peptide with one CTP amino acid peptide on the N-terminus and two CTP amino acid peptides on the C-terminus. SEQ ID NO: 28:

ATGGCCACCGGCAGCAGGACCAGCCTGCTGCTGGCCTTCGGCCTGCTGTG
CCTGCCATGGCTGCAGGAGGGCAGCGCCAGCTCTTCTTCTAAGGCTCCAC
CCCCATCTCTGCCCAGCCCCAGCAGACTGCCGGGCCCCAGCGACACACCC
ATTCTGCCCCAGTTCCCCACCATCCCCCTGAGCAGGCTGTTCGACAACGC
CATGCTGAGGGCTCACAGGCTGCACCAGCTGGCCTTTGACACCTACCAGG
AGTTCGAGGAAGCCTACATCCCCAAGGAGCAGAAGTACAGCTTCCTGCAG
AACCCCCAGACCTCCCTGTGCTTCAGCGAGAGCATCCCCACCCCCAGCAA
CAGAGAGGAGACCCAGCAGAAGAGCAACCTGGAGCTGCTGAGGATCTCCC
TGCTGCTGATCCAGAGCTGGCTGGAGCCCGTGCAGTTCCTGAGAAGCGTG
TTCGCCAACAGCCTGGTGTACGGCGCCAGCGACAGCAACGTGTACGACCT
GCTGAAGGACCTGGAGGAGGGCATCCAGACCCTGATGGGCCGGCTGGAGG
ACGGCAGCCCCAGGACCGGCCAGATCTTCAAGCAGACCTACAGCAAGTTC
GACACCAACAGCCACAACGACGACGCCCTGCTGAAGAACTACGGGCTGCT
GTACTGCTTCAGAAAGGACATGGACAAGGTGGAGACCTTCCTGAGGATCG
TGCAGTGCAGAAGCGTGGAGGGCAGCTGCGGCTTCAGCTCCAGCAGCAAG
GCCCCTCCCCCGAGCCTGCCCTCCCCAAGCAGGCTGCCTGGGCCCTCCGA
CACACCAATCCTGCCACAGAGCAGCTCCTCTAAGGCCCCTCCTCCATCCC
TGCCATCCCCCTCCCGGCTGCCTGGCCCCTCTGACACCCCTATCCTGCCT
CAG.

In one embodiment, methods comprise use of a nucleic acid sequence comprising a coding portion encoding a CTP-modified hGH disclosed herein. In another embodiment, a method disclosed herein comprises use of a nucleic acid sequence as set forth in SEQ ID NO: 28. A skilled artisan would appreciate that a nucleic acid sequence may be a part of an expression vector comprising a coding portion encoding a CTP-modified hGH disclosed herein.

In one embodiment, severe IGF-1 deficiency is defined as (i) height standard deviation score less than or equal to −3.0 and (ii) basal IGF-1 levels below the 2.5th percentile for age and gender and or below ˜3SDS.

In one embodiment, severe IGF-1 deficiency is defined as (i) height standard deviation score less than or equal to −3.0 and (ii) basal IGF-1 standard deviation score less than or equal to −3.0 and (iii) normal or elevated growth hormone (GH).

In one embodiment, the methods of administrating the CTP-modified IGF-1 or IGF-described herein result in improved compliance to IGF-1 treatment due to ease of use. In another embodiment, the methods of administrating the CTP-modified IGF-1 or IGF-described herein result in reduced dosing frequency and a better safety profile. In another embodiment, the methods of administrating the CTP-modified IGF-1 or IGF-described herein result in easier handling of the drug treatment and better compliance. In another embodiment, the methods of administrating the CTP-modified IGF-1 or IGF-described herein result in improved quality of life for the patient. In another embodiment, the methods of administrating the CTP-modified IGF-1 or IGF-described herein result in improved efficacy of the drug treatment program.

In another embodiment, the methods of administrating the CTP-modified IGF-1 or IGF-described herein result in less impact on glucose and fewer hypoglycemic incidence. In another embodiment, the methods of administrating the CTP-modified IGF-1 or IGF-described herein result in fewer injection site reaction and lipoatrophy.

According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

In alternative embodiments, the invention includes use of the recited CTP modified IGF-1 to treat the disorders listed above and further includes use of such CTP modified IGF-1 in the manufacture of a medicament to treat such diseases, conditions and disorders.

All patents, patent applications, and scientific publications cited herein are hereby incorporated by reference in their entirety.

The following examples are presented in order to more fully illustrate the preferred embodiments of the invention. They should in no way be construed, however, as limiting the broad scope of the invention.

### EXAMPLES

### Example 1: Construction of CTP-Modified IGF-1

Five CTP-modified IGF-1 variants were constructed. Different features were considered in the designing of the CTP-modified IGF1 variants, including the number and position of CTPs and the addition of the N terminal pro-peptide.

The five designed CTP-modified IGF-1 variants are described in Table 4.

TABLE 4
Summary of the CTP-modified IGF-1 Variants

| | | Active | Protein |
|---|---|---|---|
| CTP-Modified | | Protein | weight |
| Variant | Expression Product | Structure | (kDa) |
| MOD-1301-1 | SP_{hGH}-CTPx3-IGF1 | CTPx3 -IGF1 | 16.00 |
| MOD-1301-2 | SP_{IGF1}-PP_{IGF1}- | IGF1 -CTPx4 | 18.78 |
| | IGF1-CTPx4 | | |
| MOD-1301-3 | SP_{IGF1}-PP_{IGF1}- | IGF1-CTPx3 | 16.00 |
| | IGF1-CTPx3 | | |
| MOD-1301-4 | SP_{hGH}-CTP-IGF1- | CTP-IGF1- | 16.00 |
| | CTPx2 | CTPx2 | |
| MOD-1301-5 | SP_{hGH}-CTPx2-IGF1- | CTPx2-IGF1- | 24.34 |
| | CTPx4 | CTPx4 | |

Harvests of the CTP-modified variants were purified on affinity column and tested in various in vitro and in vivo assays.

### Example 2: Pharmacology Assessment Via Weight Gain Assay (WGA)

In order to evaluate the in vivo potency of the CTP-modified IGF-1 variants, weight gain assays (WGAs) were conducted in hypophysectomized rats. Although bone growth is the primary endpoint for the evaluation of the effect of IGF1, the WGA in hypophysectomized rats is common and a simpler model to evaluate the pharmaceutical activity of IGF-1. The hypophysectomized rat model is a growth-deficient nonclinical pharmacology model, which is considered to be relevant to and predictive of the bone growth response observed in response to IGF-1 therapy, as Increlex, in human clinical trials in children with primary IGFD.

All CTP-modified IGF-1 variants were injected subcutaneously to hypophysectomized rats, as a comparison to the commercial hIGF1, Increlex (Mecasermin).

A summary of the four WGAs is presented in Table 5.

TABLE 5
CTP-Modified IGF-1 Variants In-vivo Pharmacological WGA Studies

| Test | | | | | Frequency of | Dose in nmol/kg | |
|---|---|---|---|---|---|---|---|
| article | Study description | Study # | Species | ROA | admin. | (and in mg/kg) | Summary of results |
| Increlex | The study | 77735 | hypophy- | SC | MOD-1301-3 on | Increlex: 157 or 314 | 1. The Increlex vehicle |
| and | Compared the | (WGA #1) | sectomised | | day 1, | (1.2 or 2.4 mg/kg) | showed negligible activity. |
| MOD- | efficacy of | | male SPF | | Increlex and its | per injection. | 2. Only hGH showed |
| 1301-3 | Increlex on | | Sprague | | corresponding | 941 or 1883 (7.2 | significant (p-value < 0.01) |
| | body weight | | Dawley rats | | vehicle on days | or 14.4 mg/kg) weekly | differences between BWG |
| | gain relative | | of the strain | | 1-6 | accumulated dose. | on day 7 over vehicle. |
| | to its | | Crl:OFA (SD) | | | MOD-1301-3: 300 or | 3. Activity of daily Increlex |
| | corresponding | | | | | 900 (4.8 or 14.4 mg/kg) | administrations at two dose |
| | vehicle and | | | | | per injection | groups was less than the |
| | to the growth | | | | | (which is also the | activity of the hGH |
| | hormone (hGH) | | | | | accumulated dose). | reference standard. |
| | reference | | | | | | 4. Single administration at low |
| | standard. In | | | | | | dose level of MOD-1301-3 |
| | addition, | | | | | | showed negligible activity, |
| | evaluation of | | | | | | whereas the high dose level |
| | MOD-1301-3 | | | | | | showed pronounce |
| | activity | | | | | | BWG immediately |
| | was performed. | | | | | | after administration |
| | | | | | | | but didn't last 7 days. |
| Increlex | The study | 77749 | hypophy- | SC | MOD-1301 | Increlex: 314 | 1. Significant differences |
| and the | compared the | (WGA #2) | sectomised | | variants and their | (2.4 mg/kg) | (p-value < 0.01, except from |
| five | weight gain | | male SPF | | corresponding | per injection. | variant 4, which has |
| MOD- | efficacy | | Sprague | | vehicle, on days | 1883 (14.4 mg/kg) | p-value < 0.05) between |
| 1301 | between twice | | Dawley rats | | 1 and 3, | accumulated dose. | BWG on day 7 and that |
| variants | a week injections | | of the strain | | Increlex on days | MOD-1301 variants: | of the vehicle was observed |
| | of the five | | Crl:OFA (SD) | | 1-6 | 1883 (30.1 mg/kg | in all test article groups. |
| | MOD-1301 | | | | | for MOD-1301-1, | 2. The activity of twice a |
| | variants to | | | | | MOD-1301-3 | week administration of |
| | daily Increlex | | | | | and MOD-1301-4, | MOD-1301-1 was |
| | administrations | | | | | 35.3 mg/kg for | below the activity of the |
| | | | | | | MOD-1301-2 and | Increlex, whereas the activity |
| | | | | | | 45.8 mg/kg for | of MOD-1301-3, 4 and 5 |
| | | | | | | MOD-1301-5) | was comparable to the activity |
| | | | | | | per injection. 3765 | of the Increlex, and the |
| | | | | | | (60.2 mg/kg for | activity of MOD-1301-2 was |
| | | | | | | MOD-1301-1, | above the activity of the |
| | | | | | | MOD-1301-3 and | Increlex. |
| | | | | | | MOD-1301-4, | |
| | | | | | | 70.6 mg/kg | |
| | | | | | | for MOD-1301-2 and | |
| | | | | | | 91.6 mg/kg for | |
| | | | | | | MOD-1301-5) | |
| | | | | | | accumulated dose. | |
| Increlex | The study | 77888 | hypophy- | SC | MOD-1301 | Increlex: 314 | Increlex was found to be toxic, |
| and the | compared the | (WGA #3) | sectomised | | variants and their | (2.4 mg/kg) | and its treatment has been |
| five | efficacy of the | | male SPF | | corresponding | per injection for the | discontinued on |
| MOD- | five MOD-1301 | | Sprague | | vehicle, on days | first two days and | day 4 and thereafter. |
| 1301 | variants on | | Dawley rats | | 1 and 4, | then 261 (2 mg/kg), | All MOD-1301 variants had |
| variants | body weight | | of the strain | | Increlex on day | for the third day*. | significant (p-value < 0.01, |
| | gain, injected | | Crl:OFA (SD) | | 1-6 | 889 (6.8 mg/kg) | except from variant 5, |
| | in longer | | | | | accumulated dose | which has p-value < 0.05) |
| | interval, to | | | | | (as oppose to the | higher body weight gain |
| | daily Increlex | | | | | expected accumulated | then the corresponding vehicle |
| | administrations | | | | | dose of 1883 | (PBS buffer), as measured |
| | | | | | | (14.4 mg/kg)). | on Day 7. MOD-1301-2 had |
| | | | | | | MOD-1301 variants: | the highest body weight gain |
| | | | | | | 1883 (30.1 mg/kg for | during this period, followed by |
| | | | | | | MOD-1301-1, | MOD-1301-4, 3, 1 and MOD- |
| | | | | | | MOD-1301-3 and | 1301-5. |
| | | | | | | MOD-1301-4, 35.3 | |
| | | | | | | mg/kg for MOD-1301-2 | |
| | | | | | | and 45.8 mg/kg for | |
| | | | | | | MOD-1301-5) per | |
| | | | | | | injection. 3765 | |
| | | | | | | (60.2 mg/kg for MOD- | |
| | | | | | | 1301-1, MOD-1301-3 | |
| | | | | | | and MOD-1301-4, | |
| | | | | | | 70.6 mg/kg for | |
| | | | | | | MOD-1301-2 and 91.6 | |
| | | | | | | mg/kg for MOD-1301-5) | |
| | | | | | | accumulated dose. | |
| Increlex | Compare the | 77937 | hypophy- | SC | MOD-1301 | Increlex: 157 | Significant differences |
| and | efficacy for body | (WGA #4) | sectomised | | variants and their | (1.2 mg/kg) | (p-value <0.05) between |
| MOD- | weight gain of | | male SPF | | corresponding | per injection. | BWG on day 7 compared |
| 1301-2, | three most | | Sprague | | vehicle, on days | 941 (7.2 mg/ml) | to vehicle was observed |
| 3 and 5 | promised | | Dawley rats | | 1 and 4, | accumulated dose. | in the high dose groups |
| | MOD-1301 | | of the strain | | Increlex on days | MOD-1301 variants: | of MOD-1301-2 and 3. |
| | variants | | Crl:OFA (SD) | | 1-6 | 470 (7.5 mg/kg for | The groups treated with |
| | administrated | | | | | MOD-1301-3, 8.8 | MOD-1301 variants have |
| | twice a week | | | | | mg/kg for MOD-1301-2 | all shown a higher body |
| | to daily Increlex | | | | | and 11.4 mg/kg for | weight gain than the group |
| | administrations | | | | | MOD-1301-5) or 941 | treated with Increlex, except |
| | | | | | | (15 mg/kg for | from the low dose MOD- |
| | | | | | | MOD-1301-3, | 1301-5, where the body |
| | | | | | | 17.7 mg/kg for | weight gain was comparable |
| | | | | | | MOD-1301-2 and 22.9 | to the vehicle control. |
| | | | | | | mg/kg for MOD- | Variants 2 and 3 resulted |
| | | | | | | 1301-5) per injection. | with the highest body |
| | | | | | | 941 (15 mg/kg for | weight gain from Day 1 |
| | | | | | | MOD-1301-3, 17.7 | to Day 7 at the high dose (5.4 |
| | | | | | | mg/kg for MOD-1301-2 | and 6.0 g), then the high |
| | | | | | | and 22.9 mg/kg for | dose of variant 5 (3.5 g). |
| | | | | | | MOD-1301-5) or | Dose-dependent increases |
| | | | | | | 1882 (30 mg/kg for | in body weight was |
| | | | | | | MOD-1301-3, 35.3 | observed especially |
| | | | | | | mg/kg for MOD-1301-2 | in variant 3 (two folds |
| | | | | | | and 45.8 mg/kg for | increase in BWG as a result |
| | | | | | | MOD-1301-5) | of two folds increase in |
| | | | | | | accumulated dose. | administrated dose). |

*Due to lethality to animals, the Increlex dose was lowered on day 3 and stopped thereafter.

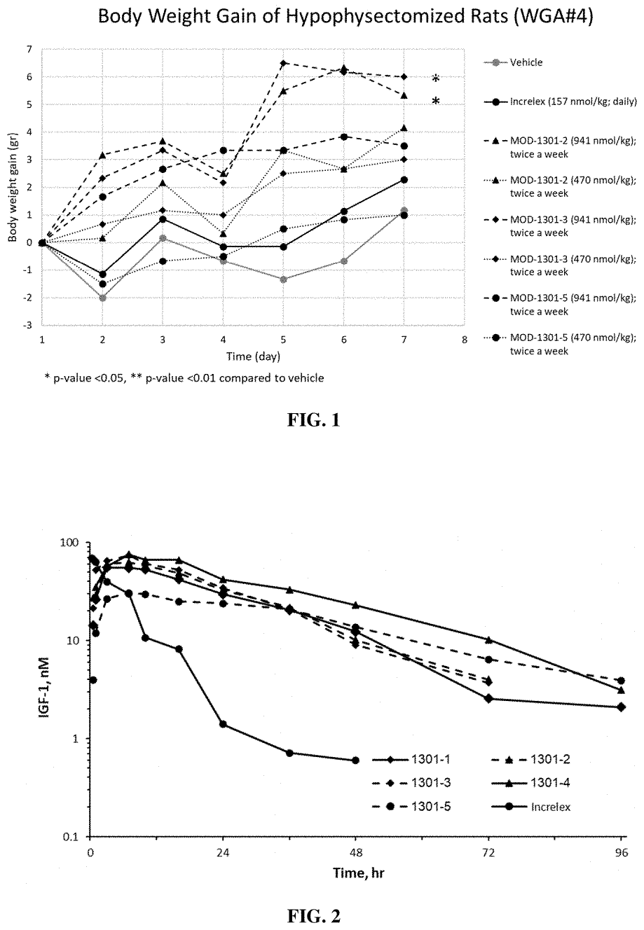

Dose-dependent increases in body weight were seen compared to control animals. In addition, the same accumulated doses of CTP-modified IGF-1 variants and Increlex achieved similar body weight gain (FIG. 1) by using only two injections instead of daily, respectively. Moreover, CTP-modified IGF-1 variants showed a better safety profile, while although Increlex administered doses were lower than the CTP-modified IGF-1 variants doses, animals were found dead only in the Increlex group.

### Example 3: Pharmacokinetic (PK) Studies

The pharmacokinetic profiles of CTP-modified IGF-1 variants administered to rats were measured in two PK studies (Study 13161 and Study 13165) following administration of a single SC injection of CTP-modified IGF-1 variants, as compared to Increlex. Serum samples were collected at several timepoints and analyzed using IGF1 R&D ELISA kit. The design of the experiments is detailed in the Table 6.

TABLE 6
MOD-1301 PK studies

| Test | Study | | | ROA & | |
|---|---|---|---|---|---|
| article | description | Study # | Species | Frequency | Dose (mg/kg) |
| Increlex | Compare the | 13161 | Healthy male | SC, Single | MOD-1301 variants: 2 mg/kg. |
| & MOD- | biological | (PK#1) | SD rats at the | injection | Increlex: 1 mg/kg (higher doses |
| 1301 | half-life of | | age of about | on day 1 | were lethal) |
| variants | MOD-1301 | | eight weeks at | | Bleeding timepoints: |
| | variants to | | study initiation | | For Increlex & MOD-1301: |
| | Increlex in | | | | Pre-dose, 1, 3, 7, 10, 16, 24, 36, |
| | rats | | | | 48, 72 |
| Increlex | Compare the | 13165 | Healthy male | SC, Single | 2 mg/kg from MOD-1301 variants |
| and | biological | (PK#2) | SD rats at the | injection | and 1 mg/kg from Increlex |
| MOD- | half-life of | | age of about | on day 1 | Bleeding timepoints: |
| 1301 | MOD-1301 | | eight weeks at | | For Increlex: Pre-dose, 0.33, 0.67, |
| variants | variants to | | study initiation | | 1, 3, 7, 10, 16, 24, 36, 48, 72 |
| | Increlex in | | | | For MOD-1301: Pre-dose, 0.5, 1, |
| | rats | | | | 3, 7, 10, 16, 24, 36, 48, 72 & 96 h |

FIG. 2 shows the average PK results of the CTP-modified IGF-1 variants versus Increlex.

The obtained half-life values of MOD-1301 variants were increased by 2.35 (for MOD-1301-3) to 4.14 (for MOD-1301-5) fold than Increlex half-life, as summarized in Table 7. Furthermore, the systemic exposure to the MOD-1301 variants was ˜4-6-fold higher, as compared to the Increlex (see the dose-normalized AUC_{0-inf} values in Table 7).

TABLE 7
Pharmacokinetic parameters of the analyzed compounds, based on non-
compartmental pharmacokinetic analysis of PK studies# 13161 & 13165

| | | Compound | | | | | |
|---|---|---|---|---|---|---|---|
| Parameter | Units | 1301-1 | 1301-2 | 1301-3 | 1301-4 | 1301-5 | Increlex |
| Dose | mg/kg | 2 | 2 | 2 | 2 | 2 | 1 |
| Dose | nmol/kg | 125 | 107 | 125 | 125 | 82 | 131 |
| Cₘₐₓ | nmol/L | 55.23 | 62.78 | 73.75 | 75.45 | 30.46 | 69.2 |
| Tₘₐₓ | h | 3 | 7 | 7 | 7 | 7 | 0.3333 |
| AUC₀₋ₜ | nmol · h/L | 1778 | 1875 | 1969 | 2756 | 1450 | 474 |
| AUC_{0-inf} | nmol · h/L | 1831 | 1965 | 2048 | 2847 | 1595 | 479 |
| k | 1/h | 0.0396 | 0.0444 | 0.0471 | 0.0344 | 0.0268 | 0.1108 |
| t_{1/2} | h | 17.51 | 15.63 | 14.72 | 20.12 | 25.86 | 6.25 |
| AUMC_{0-inf} | nmol · h²/L | 48785 | 48251 | 46631 | 89528 | 66730 | 3707 |
| MRT_{0-inf} | h | 26.64 | 24.56 | 22.77 | 31.45 | 41.83 | 7.74 |
| V/F | L/kg | 1.725 | 1.223 | 1.298 | 1.276 | 1.923 | 2.462 |
| CL/F | L/kg/h | 0.068 | 0.054 | 0.061 | 0.044 | 0.052 | 0.273 |
| AUC_{0-inf/Dose} | kg · h/L | 14.6 | 18.4 | 16.4 | 22.8 | 19.4 | 3.7 |

### Example 4: In Vitro Potency Using Cell Based Assay (CBA)

Cell based assay was used in order to evaluate the in vitro potency of MOD-1301 variants, while measuring the variants ability to stimulate the IGF-1 receptor, as compared to hIGF1.

For this aim, the following kit of DISCOVERX was used: PathHunter, IGF1R Bioassay Kit, Catalog No. 93-0505Y1 Series. This kit included cells that over expressed recombinant IGF1 receptor fused to one fragment of β-galactosidase (β-gal) enzyme. The second part of β-gal was fused to SH2 protein which binds to the activated receptor. Upon activation (by IGF1 binding), the SH2 fusion protein were bound to the phosphorylated receptor, forcing complementation of the two parts of β-gal to form an active β-gal enzyme. β-gal enzymatic activity translated into luminesces that was quantitatively measured.

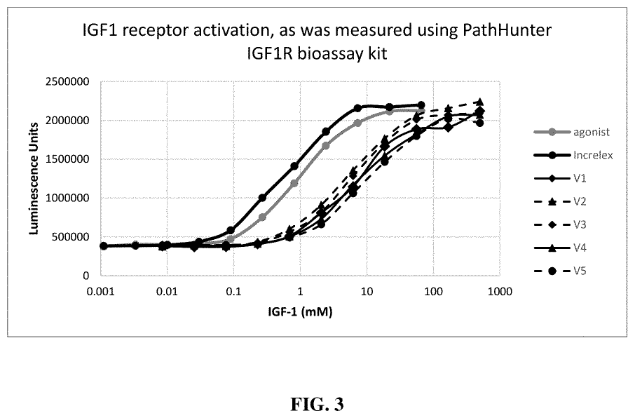

FIG. 3 shows a representative CBA results of IGF1 receptor activation by Increlex and MOD-1301 variants from one of four CBAs that were performed with all MOD-1301 variants. As expected, due to the addition of CTP copies, the IGF-1 CTP variants resulted with reduced potency compare to Increlex which can be seen from the right shifted curves of IGF1 variants. The resulted higher EC50 was calculated using Prism software and is summarized in Table 8. MOD-1301-2 was the most potent MOD-1301 variant, with EC₅₀ of 4.2 nM, which is an -9-fold reduction in the receptor activation, as compare to Increlex. Variants MOD-1301-2 and 3 showed very similar potency, while MOD-1301-5, was the less potent variant, with a ˜21-fold reduction in the IGF-1R stimulation potency, as compare to Increlex. From a potency perspective, CTP(s) in the C terminal is preferred, while CTP(s) on both sides causes poor IGF1R stimulation potency.

TABLE 8
CBA Summary

| | | | | | | Average | | | |
|---|---|---|---|---|---|---|---|---|---|
| | | | | | | | fold reduction, | Statistic | |
| | | EC₅₀ | | | | | as compare to | | % |
| Variant | Structure | CBA#1 | CBA#2 | CBA#3 | CBA#5 | EC₅₀ | Increlex | Stdev | CV |
| Increlex | IGF1 | 0.22 | 0.54 | 0.59 | 0.52 | 0.47 | 1 | 0.17 | 35.6 |
| Agonist | IGF1 | 0.67 | 0.70 | 0.96 | 1.01 | 0.83 | 1.8 | 0.17 | 20.9 |
| V1 | CTP × 3-IGF1 | 5.76 | 4.32 | 6.60 | 8.14 | 6.20 | 13.3 | 1.60 | 25.7 |
| (MOD-1301-1) | | | | | | | | | |
| V2 | IGF1-CTP × 4 | 4.38 | 2.63 | 5.62 | 4.22 | 4.21 | 9.0 | 1.23 | 29.1 |
| (MOD-1301-2) | | | | | | | | | |
| V3 | IGF1-CTP × 3 | 5.78 | 3.86 | 5.75 | 5.52 | 5.23 | 11.2 | 0.92 | 17.5 |
| (MOD-1301-3) | | | | | | | | | |
| V4 | CTP-IGF1- | 5.96 | 7.83 | 8.17 | 8.82 | 7.70 | 16.5 | 1.23 | 16.0 |
| (MOD-1301-4) | CTP × 2 | | | | | | | | |
| V5 | CTP × 2-IGF1- | 6.98 | 7.09 | 9.37 | 15.60 | 9.76 | 20.9 | 4.05 | 41.5 |
| (MOD-1301-5) | CTP × 4 | | | | | | | | |

MOD-1301-2 and MOD-1301-3, the two most potent variants (See Table 8), were chosen for further development work. The two variants were purified from stably expressing cells, and were tested by CBA compared to Increlex. As shown in Table 9, both variants displayed similar potency with EC₅₀ of 2.41 nM for MOD-1301-2 and 1.82 nM for MOD-1301-3, an average of only ˜3-fold reduction in receptor activation, as compared to Increlex.

TABLE 9
CBA summary for the selected clone of MOD-1301 variants

| | | | | | | Average | | | |
|---|---|---|---|---|---|---|---|---|---|
| | | EC₅₀ | | | | | Fold reduction, | | |
| | | CBA | CBA | CBA | CBA | | as compare to | Statistic | |
| Variant | Structure | #8.1 | #8.2 | #9.1 | #9.2 | EC₅₀ | Increlex | Stdev | % CV |
| Increlex | IGF1 | 0.64 | 0.82 | 0.61 | 0.66 | 0.68 | 1.00 | 0.09 | 13.83 |
| MOD-1301-2 | IGF1-CTP × 4 | 2.39 | 1.63 | 2.64 | 2.98 | 2.41 | 3.53 | 0.57 | 23.79 |
| MOD-1301-3 | IGF1-CTP × 3 | 1.90 | 1.76 | 1.88 | 1.76 | 1.82 | 2.67 | 0.08 | 4.20 |

### Example 5: Binding Affinity of MOD-1301 to IGFBP3 Using BIAcore

Interactions of IGF1 variants with the carrier protein IGFBP3 were determined using BIAcore. BIAcore is based on surface plasmon resonance technology, which allows detection of biomolecular interactions. IGFBP3 (rhIGFBP3, R&D, #675-B3-025) was immobilized to the sensor surface, and Increlex or MOD-1301 variants were injected one after the other over the surface. Interactions between IGFBP3 and IGF1 variants, if occurring, were detected. As shown in Table 10, the addition of CTP units increases the KD (the “equilibrium dissociation constant”), which is the IGF1 concentration where half of the binding sites of IGFBP3s are occupied. This reflects decreased affinity between MOD-1301 Variants and IGFBP3, as compared to Increlex. The binding affinities measurement of MOD-1301 variants to IGFBP3 was performed as part of in vitro MOD-1301 variants characterization. It was done to assess the potential interference of the CTPs to IGF-1 BP binding, and to be able to select the variant with the lower reduction of the affinities to the IGFBP3

Among the MOD-1301 variants, MOD-1301-2 and MOD-1301-3, with CTPs at the C terminal, showed the highest affinity to IGFBP3, which is -10-12.8 time less than Increlex affinity. MOD-1301-1, possesses CTP copies at its N terminal, and has intermediate affinity, while MOD-1301-4 and MOD-1301-5, with CTP copies on both N and C terminal sides, showed the lowest affinity, ˜21 times less than Increlex. The observation that the C terminal CTP variants (MOD-1301-2 and MOD-1301-3) showed better affinity to the IGFBP3 was expected, as the binding site for IGFBP3 is near the N terminal of the IGF-1 molecule (Denley, Adam, et al. “Molecular interactions of the IGF system.” Cytokine & growth factor reviews 16.4-5 (2005): 421-439). According to Denley et. al, modifications of critical residues near the N terminal of the IGF-1 sequence can reduce binding to IGFBP3 by more than 1,000 fold. However, although the interaction with IGFBP3 was interrupted by adding the CTP copies at the N terminal for MOD-1301 variants 1, 4 and 5, the reduced affinity was only by one order of magnitude compared to Increlex, and no more than two-fold, as compared to the C terminal CTP variants.

TABLE 10
Summary of BIAcore parameters

| | | | | | Average | | | |
|---|---|---|---|---|---|---|---|---|
| | | KD (nM) | | | | Fold reduction, | Statistic | |
| | | BIAcore | BIAcore | BIAcore | KD | as compared to | | % |
| Variant | Structure | #a | #b | #c | (nM) | Increlex | Stdev | CV |
| Increlex | IGF1 | 0.20 | N/A | 0.18 | 0.19 | 1.0 | 0.01 | 6.8 |
| MOD-1301-1 | CTP × 3-IGF1 | 4.07 | 2.57 | 1.98 | 2.87 | 15.3 | 1.08 | 37.5 |
| MOD-1301-2 | IGF1-CTP × 4 | 2.56 | 1.67 | 1.33 | 1.85 | 9.9 | 0.64 | 34.5 |
| MOD-1301-3 | IGF1-CTP × 3 | 4.08 | 1.72 | 1.39 | 2.40 | 12.8 | 1.47 | 61.2 |
| MOD-1301-4 | CTP-IGF1-CTP × 2 | 4.94 | 3.42 | 2.53 | 3.63 | 19.4 | 1.22 | 33.5 |
| MOD-1301-5 | CTP × 2-IGF1-CTP × 4 | 5.77 | 3.84 | 2.80 | 4.14 | 22.1 | 1.51 | 36.4 |

### Example 6: Safety Evaluation Using Hypoglycemic Assays and CBA

IGF1 is structurally related to insulin, therefore it binds and stimulates the insulin receptor, although at lower affinity than insulin, but still can cause mild or severe hypoglycemia events in animals and humans. 42% of patients treated with the commercial IGF1(Increlex), reported hypoglycemia events at least once during their course of therapy. This is a major safety issue; thus, it was important to explore the potential effect of MOD-1301 variants on blood glucose, relative to Increlex.

The WGA studies showed a disparity between Increlex and MOD-1301 variants regarding the safety profile, probably due to effect on blood glucose levels. Animals from the Increlex groups in each one of the four WGAs, were found dead during the assays, while no deaths occurrence were in the MOD-1301 groups, although similar or higher doses (6 fold) of MOD-1301 variants were administrated.

In order to assess directly the hypoglycemic effects of Increlex, as compared to MOD-1301 variants, the fasted normal rat model was chosen. Table 11 summaries the hypoglycemic studies that were performed, where Increlex or MOD-1301-2 or MOD-1301-3 were injected once to fasted rats and blood glucose was measured up to 12 hours post dose.

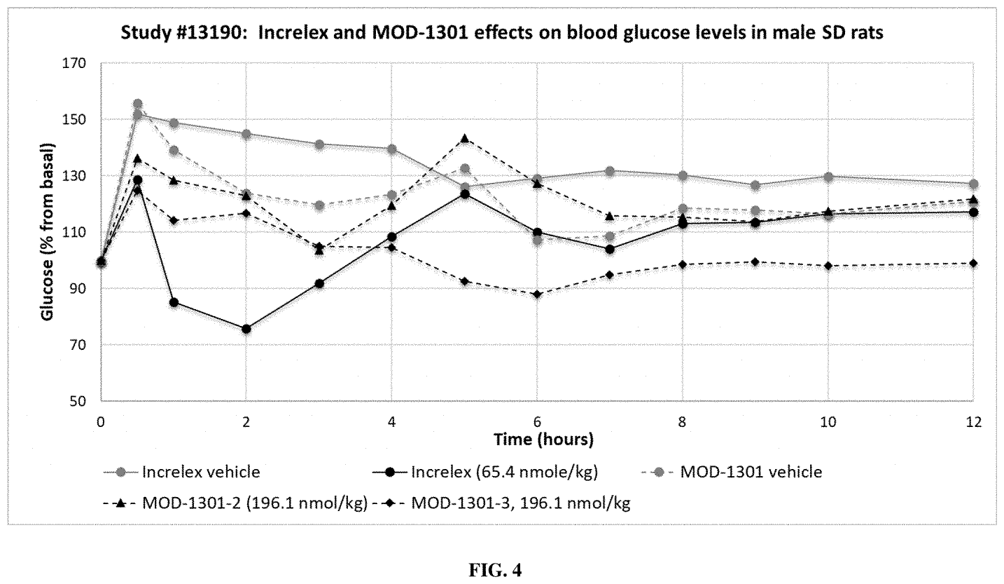

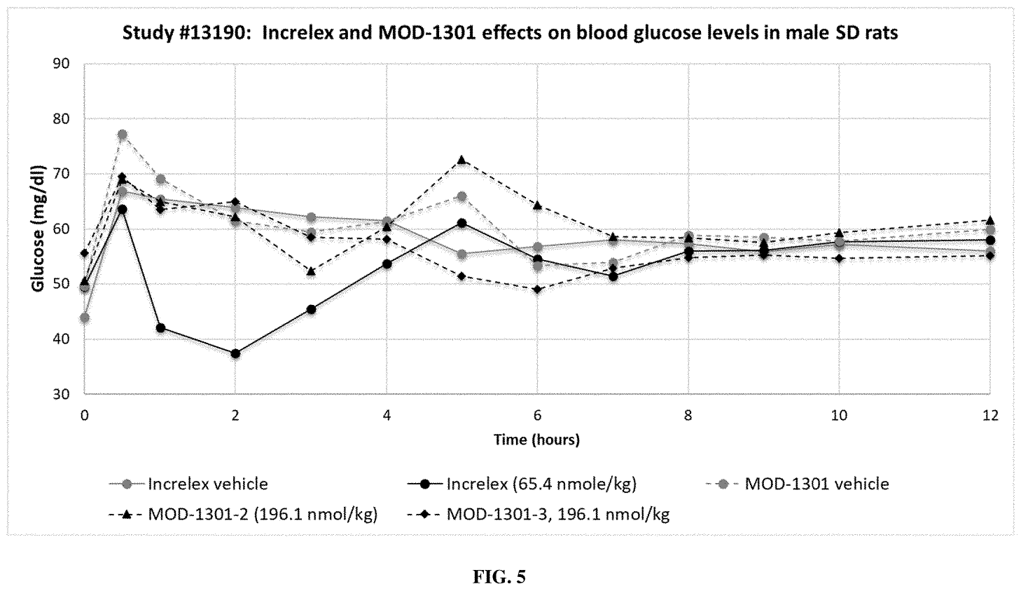

As shown in FIG. 4, while 0.5 mg/kg (65.4 nmol/kg) of Increlex caused a significant decrease of 24% in blood glucose level (as was determined by T-TEST between the glucose levels in the time where the minimal glucose levels were observed and the basal level of this group), MOD-1301 variants, at ˜7-fold higher dose (3.14-3.68 mg/kg, 196.1 nmol/kg), did not cause for any decrease (MOD-1301-2) or only a small, insignificant decrease, of 12% (MOD-1301-3) in blood glucose, 6 hours post injection (see assay #13190 in Table 11).

FIGS. 4 & 5 show blood glucose levels (% from basal and actual concentrations, respectively) following injection of Increlex or MOD-1301 variants.

TABLE 11
Summary of hypoglycemic assays

| Study | | Hypoglycemic | Dose in nmol/kg | |
|---|---|---|---|---|
| # | Test article | model | (and in mg/kg) | Results |
| 13188 | Increlex and | fasted SD male | Increlex at | While 65.4 nmol/kg (0.5 mg/kg) |
| | MOD-1301-3 | rats | 65.4 nmol/kg | of Increlex caused immediately |
| | | | (0.5 mg/kg) | for a 35% significantly (p-value < |
| | | | and MOD-1301 at | 0.01) decrease in blood glucose, |
| | | | equal molar dose | surprisingly MOD-1301-3, at 6 |
| | | | (65.4 nmol/kg, | fold higher mg/kg dose, cause |
| | | | 1.05 mg/kg) and | for an un-significant decrease |
| | | | 6-fold higher | of 15%, 6 hours post injection. |
| | | | mg/kg dose | |
| | | | (196.1 nmol/kg, | |
| | | | 3.14 mg/kg) | |
| 13190 | Increlex, | fasted SD male | Increlex at | While 65.4 nmol/kg (0.5 mg/kg) |
| | MOD-1301-2 | rats | 65.4 (0.5 mg/kg) | of Increlex cause 24% decrease |
| | and MOD- | | and MOD-1301-2 | (p-value < 0.01) in blood glucose |
| | 1301-3 | | and MOD-1301-3 | level, MOD-1301 variants, |
| | | | at ~7-fold | in ~7-fold higher mg/kg dose, |
| | | | higher dose | surprisingly didn't cause |
| | | | mg/kg, 196.1 | for any decrease (variant #2) |
| | | | (3.14-3.68 | or only to an un-significant |
| | | | nmol/kg) | decrease of 12% (variant #3). |

Table 12 presents Increlex and MOD-1301-2/MOD-1301-3 effective doses at the WGAs in hypophysectomized rats, as compared to the doses tested in the fasted normal rat hypoglycemic model. The usage of the same dose ratio for both Increlex and MOD-1301-2/MOD-1301-3 (last column, Table 12) between the models, strengthen the conclusion that Increlex effective dose causes for a decrease in blood glucose levels (and hypoglycemia as a result), while MOD-1301-2/MOD-1301-3 resulted to be safer from that perspective.

TABLE 12
Increlex and MOD-1301-2/MOD-1301-3 effective doses (dose
per injection) at the WGA, as compared to the doses
tested in the fasted hypoglycemic normal rat model

| | Dose | Dose | Dose |
|---|---|---|---|
| | for hypoglycemic | for WGA | ratio of |
| Test article | assay (nmol/kg) | (nmol/kg) | WGA/Hypo |
| Increlex | 65.4 | 314 | 4.8 |
| MOD-1301-2/ | 196.1 | 941 | 4.8 |
| MOD-1301-3 | | | |

In addition to the in-vivo model, CBAs have been performed to assess the potency of MOD-1301-2 and MOD-1301-3 in stimulating the insulin receptor (Table 13). As detailed in Table 13, MOD-1301 variants showed a lower affinity (higher EC₅₀ values, ˜13 fold higher for variant 3, and 19.5 fold higher for variant 2) to the insulin receptor, as compared to Increlex.

TABLE 13
Affinity (EC₅₀) of MOD-1301 variants to the insulin receptor, as compare to Increlex

| | | | | | | | | Affinity fold | | |
|---|---|---|---|---|---|---|---|---|---|---|
| | | | | | | | | reduction vs | | % |
| EC50 (nM) | CBA#1 | CBA#2 | | CBA#3 | | | Average | Increlex | Stdev | CV |
| Increlex | 13.0 | 15.6 | 16.4 | 13.9 | 15.0 | 15.8 | 14.9 | 1.0 | 1.3 | 8.5 |
| MOD-1301-2 | 237.6 | 514.5 | 199.5 | 176.7 | 331.8 | N/A | 292.0 | 19.5 | 137.7 | 47.2 |
| MOD-1301-3 | 211.9 | 82.7 | 164.4 | 199.4 | 340.0 | N/A | 199.7 | 13.4 | 93.2 | 46.7 |

Two additional CBAs were performed with MOD-1301-2 and MOD-1301-3, purified from stably expressing cells, as compared to Increlex. As can be seen in Table 14, both variants showed much lower potential to stimulate insulin receptors than Increlex (11-fold reduction), while the IGF-1 receptor stimulation is only ˜3-fold lower (Table 9 and Table 15). These results strengthen the conclusion that MOD-1301 variants possess higher safety profile compare to Increlex, with a lower potential to stimulate insulin receptor (˜11 fold lower) than Increlex, while the IGF-1 receptor stimulation is only 3 fold lower.

The calculated ratios between the EC₅₀ to Insulin receptor and IGF-1 receptors was much higher in the MOD-1301 variants, which reflect the lower potential of MOD-1301 variants to stimulate the Insulin receptor over the IGF-1 receptors, compared to Increlex.

TABLE 14
Insulin receptor CBA summary for clone selected MOD-1301 variants

| | CBA | CBA | CBA | CBA | | Fold | | % |
|---|---|---|---|---|---|---|---|---|
| | #4.1 | #4.2 | #5.1 | #5.2 | Average | reduction | Stdev | CV |
| Increlex | 27.3 | 28.6 | 28.5 | 28.9 | 28.3 | 1.0 | 0.7 | 2.5 |
| MOD- | 508.6 | 430.3 | 313.5 | 296.1 | 387.1 | 13.7 | 100.5 | 26.0 |
| 1301-2 | | | | | | | | |
| MOD- | 260.4 | 258.3 | 169.5 | 207.8 | 224.0 | 7.9 | 43.7 | 19.5 |
| 1301-3 | | | | | | | | |

TABLE 15
Comparison between IGF-1R vs. Insulin receptor stimulation
by MOD-1301 variants 2, 3 and Increlex

| | | | | Average Fold |
|---|---|---|---|---|
| | Average EC₅₀ (nM) | | | stimulation |
| | | MOD- | MOD- | reduction |
| | Increlex | 1301-2 | 1301-3 | (MOD/Increlex) |
| Insulin Receptor | 28.3 | 387.1 | 224.0 | ~11 |
| IGF-1 Receptor | 0.68 | 2.41 | 1.82 | ~3 |
| Ratio (EC₅₀ | 42 | 161 | 123.0 | |
| Insulin receptor/ | | | | |
| EC₅₀ IGF-1 | | | | |
| recentor) | | | | |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A polypeptide comprising a CTP-modified insulin-like growth factor 1 (IGF-1) or CTP-modified IGF-1 variant, said CTP-modified IGF-1 or IGF-1 variant comprising at least one chorionic gonadotrophin carboxy terminal peptide (CTP) attached to the amino terminus or carboxy terminus of said IGF-1 or IGF-1 variant.

2. The polypeptide of claim 1, wherein said IGF-1 is human IGF-1.

3. The polypeptide of claim 1, wherein the amino acid sequence of said IGF-1 is set forth in SEQ ID NO: 1.

4. The polypeptide of claim 1, wherein said IGF-1 variant comprises an alanine, a glycine, or a serine substitution of the amino acid residue at position 16, 25, or 49 of native sequence human IGF-1, or an alanine, a glycine, or a serine substitution of the amino acid residues at positions 3 and 49 of native sequence human IGF-1.

5. The polypeptide of claim 1, wherein said IGF-1 variant comprises a replacement of an amino acid residue located at a single position selected from the group consisting of positions 4, 5, 7, 10, 14, 17, 23, 24, and 43 of native-sequence human IGF-1 with an alanine residue.

6. The polypeptide of claim 1, wherein said IGF-1 variant comprises variant comprises a replacement of an amino acid residue at positions 1 and 70 of native-sequence human IGF-1 with a serine residue and a valine residue, respectively

7. The polypeptide of claim 6, wherein said IGF-1 variant further comprises a replacement of an amino acid residue at a single position selected from the group consisting of positions 3, 4, 5, 7, 10, 14, 17, 23, 24, 25, and 43 of native-sequence human IGF-1 with an alanine residue.

8. The polypeptide of any of claims 1 to 7, further comprising at least three CTPs attached to said IGF-1 or IGF-1 variant.

9. The polypeptide of claim 8, wherein one CTP is attached to the amino terminus of said IGF-1 or IGF-1 variant, and two CTPs are attached to the carboxy terminus of said IGF-1 or IGF-1 variant.

10. The polypeptide of claim 9, wherein the amino acid sequence of said CTP-modified IGF-1 is set forth in SEQ ID NO: 18.

11. The polypeptide of claim 8, wherein three CTPs are attached to the amino terminus of said IGF-1 or IGF-1 variant, and no CTPs are attached to the carboxy terminus of said IGF-1 or IGF-1 variant.

12. The polypeptide of claim 11, wherein the amino acid sequence of said CTP-modified IGF-1 is set forth in SEQ ID NO: 15.

13. The polypeptide of claim 8, wherein no CTPs are attached to the amino terminus of said IGF-1 or IGF-1 variant, and three CTPs are attached to the carboxy terminus of said IGF-1 or IGF-1 variant.

14. The polypeptide of claim 13, wherein the amino acid sequence of said CTP-modified IGF-1 is set forth in SEQ ID NO: 17.

15. The polypeptide of any of claims 1 to 14, further comprising at least four CTPs attached to said IGF-1 or IGF-1 variant.

16. The polypeptide of claim 15, wherein no CTPs are attached to the amino terminus of said IGF-1 or IGF-1 variant, and four CTPs are attached to the carboxy terminus of said IGF-1 or IGF-1 variant.

17. The polypeptide of claim 16, wherein the amino acid sequence of said CTP-modified IGF-1 is set forth in SEQ ID NO: 16.

18. The polypeptide of any of claims 1 to 17, further comprising at least six CTPs attached to said IGF-1 or IGF-1 variant.

19. The polypeptide of claim 18, wherein two CTPs are attached to the amino terminus of said IGF-1 or IGF-1 variant, and four CTPs are attached to the carboxy terminus of said IGF-1 or IGF-1 variant.

20. The polypeptide of claim 19, wherein the amino acid sequence of said CTP-modified IGF-1 is set forth in SEQ ID NO: 19.

21. The polypeptide of any of claims 1 to 20, wherein the amino acid sequence of any of said CTPs is set forth in SEQ ID NO: 6 or SEQ ID NO: 7.

22. The polypeptide of any of claims 1 to 20, wherein the amino acid sequence of any of said CTPs consists of a partial sequence of SEQ ID NO: 6 or SEQ ID NO: 7.

23. The polypeptide of any of claims 1 to 22, wherein said CTP-modified IGF-1 or IGF-1 variant further comprises a signal peptide at the amino terminus.

24. The polypeptide of claim 23, wherein said signal peptide is the signal peptide of IGF-1 (“SP_{IGF1}”).

25. The polypeptide of claim 24, consisting of the structure SP_{IGF1}-(CTP-Modified IGF-1 or CTP-modified IGF-1 variant).

26. The polypeptide of any of claims 24 to 25, wherein said IGF-1 signal peptide is set forth in SEQ ID NO: 2.

27. The polypeptide of claim 23, wherein said signal peptide is the signal peptide of human growth hormone (“SP_{hGH}”).

28. The polypeptide of claim 27, consisting of the structure SP_{hGH}-(CTP-Modified IGF-1 or CTP-modified IGF-1 variant).

29. The polypeptide of any of claims 27 to 28, wherein said hGH signal peptide is set forth in SEQ ID NO: 9.

30. The polypeptide of any of claims 27 to 29, wherein the amino acid sequence of said polypeptide is set forth in SEQ ID NO: 10, SEQ ID NO: 13, or SEQ ID NO: 14.

31. The polypeptide of any of claims 23 to 30, further comprising a propeptide at the carboxy terminus of said signal peptide.

32. The polypeptide of claim 31, wherein said propeptide is the first propeptide of IGF-1 (“PP_{IGF1}”).

33. The polypeptide of any of claims 31 to 32, wherein the amino acid sequence of said propeptide is set forth in SEQ ID NO: 3.

34. The polypeptide of any of claims 31 to 33, wherein said polypeptide consists of the structure SP_{IGF1}-PP_{IGF1}-(CTP-Modified IGF-1 or CTP-modified IGF-1 variant).

35. The polypeptide of any of claims 31 to 34, wherein the amino acid sequence of said polypeptide is set forth in SEQ ID NO: 11 or SEQ ID NO: 12.

36. The polypeptide of any of claims 1 to 35, wherein said IGF-1 or IGF-1 variant does not contain an E peptide.

37. The polypeptide of any of claims 1 to 36, wherein at least one CTP is glycosylated.

38. The polypeptide of any of claims 1 to 37, wherein said CTP-modified IGF-1 or IGF-1 variant binds to an Insulin receptor with an average EC₅₀ value of between 100 nM and 400 nM.

39. The polypeptide of any of claims 1 to 38, wherein said CTP-modified IGF-1 or IGF-1 variant binds to an IGF-1 receptor with an average EC₅₀ value of between 1 nM and 3 nM.

40. The polypeptide of any of claims 38 to 39, wherein the average EC₅₀ value of said Insulin receptor and said IGF-1 receptor (EC₅₀ Insulin receptor/EC₅₀ IGF-1 receptor) are present in a ratio of between 30 to 400.

41. The polypeptide of claim 40, wherein said ratio is 50, 60, 70, 80, 90, 100, 110, 120, 130, 140, or 150.

42. A CTP-modified insulin-like growth factor 1 (IGF-1) polypeptide wherein no chorionic gonadotrophin carboxy terminal peptides (CTPs) are attached to the amino terminus of said IGF-1, and three or four CTPs are attached to the carboxy terminus of said IGF-1, wherein the average EC₅₀ value of said Insulin receptor and said IGF-1 receptor (EC₅₀ Insulin receptor/EC₅₀ IGF-1 receptor) are present in a ratio of between 30 to 400.

43. The polypeptide of claim 42, wherein the amino acid sequence of said CTP-modified IGF-1 is set forth in SEQ ID NO: 11, SEQ ID NO: 12, SEQ ID NO: 16, or SEQ ID NO: 17.

44. The polypeptide of any of claims 42 to 43, wherein said ratio is 50, 60, 70, 80, 90, 100, 110, 120, 130, 140, or 150.

45. A pharmaceutical composition comprising the polypeptide of any of claims 1 to 44.

46. A dosage form comprising a pharmaceutically effective amount of a polypeptide according to any of claims 1 to 44.

47. An injectable formulation for once or twice a week administration comprising a polypeptide according to any of claims 1 to 44 and a liquid vehicle.

48. A polynucleotide encoding the polypeptide of any of claims 1 to 44.

49. The polynucleotide according to claim 48, wherein the nucleotide sequence of said polynucleotide consists any of SEQ ID NOs: 20 to 24.

50. A method of treating a human patient having an IGF-1 related disease or disorder comprising administering a pharmaceutically effective amount of the polypeptide according to any of claims 1 to 44.

51. The method of any claim 50, wherein said disease or disorder is selected from the group consisting of hyperglycemic disorder, a renal insufficiency, congestive heart failure, hepatic failure, poor nutrition, a wasting syndrome, and a catabolic state.

52. The method of claim 51, where said renal insufficiency is chronic renal failure or acute renal failure.

53. The method of claim 50, wherein said disease or disorder is IGF-1 deficiency, severe primary IGF deficiency (SP IGFD), severe primary IGF-1 deficiency (Primary IGFD), growth failure with severe primary IGF-1 deficiency, growth hormone (GH) gene deletion, mutation in the GH receptor (GHR), GH gene deletion resulting in neutralizing antibodies to GH, post-GHR signaling pathway, or IGF1 gene defects.

54. The method of any of claims 50 to 53, wherein said patient has developed neutralizing antibodies to growth hormone or has IGF-1 gene defects.

55. The method of any of claims 50 to 54, wherein said CTP modified IGF-1 or IGF-1 variant has reduced hypoglycemic side effects relative to an equal molar dose of the identical IGF-1 antagonist without said CTP modification.

56. The method of claim 55, wherein the amino acid sequence of said IGF-1 antagonist consists of SEQ ID NO: 1.

57. The method of any of claims 50 to 56, further comprising administering hGH or an insulin-like growth factor binding protein (“IGFBP”).

58. The method of claim 57, wherein said IGFBP is IGFBP-3.

59. The method of any of claims 50 to 58, wherein said patient is a child or an adult.

60. The method of any of claims 50 to 59, wherein said patient experiences improved compliance to IGF-1 treatment due to ease of use, reduced dosing frequency, or an increase in the safety profile of said CTP-modified IGF-1 or IGF-1 variant.

61. The polypeptide of any of claims 1 to 44, wherein following administration of said CTP-modified IGF-1 or IGF-1 variant to a human patient in need of treatment thereof, said patient has no more than a 15% decrease in blood glucose.

62. A method of manufacturing the polypeptide according to any of claims 1 to 44, the method comprising the steps of
(a) stably transfecting a predetermined number of cells with an expression vector comprising a coding portion encoding said polypeptide;
(b) wherein said transfected cells express and secrete said polypeptide;
(c) obtaining cell clones that overexpress said polypeptide;
(d) expanding said clones in solution to a predetermined scale;
(e) harvesting said solution containing said clones;
(f) filtering said solution containing said clones to obtain a clarified harvest solution containing said polypeptide; and,
(g) purifying and activating said polypeptide from said clarified harvest solution to obtain a purified protein solution having a desired concentration of the polypeptide;
thereby manufacturing a CTP-modified IGF-1 or IGF-1 variant polypeptide.

63. A combination comprising a therapeutically effective amount of the polypeptide according to any of claims 1 to 44 and a therapeutically effective amount of an active ingredient selected from the group consisting of human growth hormone (HGH), estrogen hormone, and IGF binding protein (“IGFBP”).

64. The combination of claim 63, wherein said IGFBP is IGFBP-3.

65. A composition comprising the polypeptide according to any of claims 1 to 44 and a composition selected from the group consisting of human growth hormone, an IGF binding protein (“IGFBP’), an estrogen hormone, or any combination thereof for use in treating an IGF-1 related disease, disorder or condition.

66. The therapeutic regimen of claim 65, wherein said IGFBP is IGFBP-3.


SEQUENCE LISTING (28 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 21 aa, Homo sapiens
MGKISSLPTQLFKCCFCDFLK

SEQ ID NO: 3 - protein, 27 aa, Homo sapiens
VKMHTMSSSHLFYLALCLLTFTSSATA

SEQ ID NO: 4 - protein, 77 aa, Homo sapiens
RSVRAQRHTDMPKTQKYQPPSTNKNTKSQRRKGWPKTHPGGEQKEGTEASLQIRGKKKEQRREIGSRNAECRGKKGK

SEQ ID NO: 5 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 6 - protein, 33 aa, Artificial Sequence
  CTP sequence
DPRFQDSSSSKAPPPSLPSPSRLPGPSDTPILQ

SEQ ID NO: 7 - protein, 28 aa, Artificial Sequence
  CTP sequence
SSSSKAPPPSLPSPSRLPGPSDTPILPQ

SEQ ID NO: 8 - protein, 12 aa, Artificial Sequence
  Truncated CTP sequence
SSSSKAPPPSLP

SEQ ID NO: 9 - protein, 26 aa, Homo sapiens
MATGSRTSLLLAFGLLCLPWLQEGSA

SEQ ID NO: 10 - protein, 180 aa, Artificial Sequence
  CTP-modified IGF-1, MOD-1301-1
MATGSRTSLLLAFGLLCLPWLQEGSASSSSKAPPPSLPSPSRLPGPSDTPILPQSSSSKAPPPSLPSPSRLPGPSDTPILPQSSSSKAPPPSLPSPSRLPGPSDTPILPQGPETLCGAELVDALQFVCGDRGFYFNKPTGYGSSSRRAPQTGIVDECCFRSCDLRRLEMYCAPLKPAKSA

SEQ ID NO: 11 - protein, 230 aa, Artificial Sequence
  CTP-modified IGF-1, MOD-1301-2
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 12 - protein, 202 aa, Artificial Sequence
  CTP-modified IGF-1, MOD-1301-3
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 13 - protein, 180 aa, Artificial Sequence
  CTP-modified IGF-1, MOD-1301-4
MATGSRTSLLLAFGLLCLPWLQEGSASSSSKAPPPSLPSPSRLPGPSDTPILPQGPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQ

SEQ ID NO: 14 - protein, 264 aa, Artificial Sequence
  CTP-modified IGF-1, MOD-1301-5
MATGSRTSLLLAFGLLCLPWLQEGSASSSSKAPPPSLPSPSRLPGPSDTPILPQSSSSKAPPPSLPSPSRLPGPSDTPILPQGPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 15 - protein, 154 aa, Artificial Sequence
  Mature CTP-modified IGF-1, MOD-1301-1 without the signal peptide
SSSSKAPPPSLPSPSRLPGPSDTPILPQSSSSKAPPPSLPSPSRLPGPSDTPILPQSSSSKAPPPSLPSPSRLPGPSDTPILPQGPETLCGAELVDALQFVCGDRGFYFNKPTGYGSSSRRAPQTGIVDECCFRSCDLRRLEMYCAPLKPAKSA

SEQ ID NO: 16 - protein, 182 aa, Artificial Sequence
  Mature CTP-modified IGF-1, MOD-1301-2 without the signal peptide or propeptide
GPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 17 - protein, 154 aa, Artificial Sequence
  Mature CTP-modified IGF-1, MOD-1301-3 without the signal peptide or propeptide
GPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 18 - protein, 154 aa, Artificial Sequence
  Mature CTP-modified IGF-1, MOD-1301-4 without the signal peptide
SSSSKAPPPSLPSPSRLPGPSDTPILPQGPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQ

SEQ ID NO: 19 - protein, 238 aa, Artificial Sequence
  Mature CTP-modified IGF-1, MOD-1301-5 without the signal peptide
SSSSKAPPPSLPSPSRLPGPSDTPILPQSSSSKAPPPSLPSPSRLPGPSDTPILPQGPETLCGAELVDALQFVCGDRGFYFNKPTGYGSSSRRAPQTGIVDECCFRSCDLRRLEMYCAPLKPAKSASSSSKAPPPSLPSPSRLPGPSDTPILPQSSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 20 - DNA, 562 bp, Artificial Sequence
  CTP-modified IGF-1, MOD-1301-1
gcggccgccatggccaccgggagccggacatctctgctgctggctttcggtctgctgtgcctgccatggctgcaggagggcagtgcttccagctctagtaaggcaccccctccatcactgccttccccttctagactgcctggaccatctgacaccccaatcctgcctcagtcatccagctctaaagctccccctccatctctgccttctccaagtcgtctgcccgggcctagtgatacaccaattctgccccagagttcatccagcaaggcaccccctccaagcctgccatcaccatccaggctgccaggcccatctgacactcctatcctgccacagggacctgagaccctgtgcggagcagaactggtggacgccctgcagttcgtctgtggggatagaggtttctactttaacaaacccacaggctatggatctagttcaaggcgggcacctcagactggcattgtggacgagtgctgttttaggtcctgcgatctgagacgcctggaaatgtactgtgcccctctgaagccagccaaatccgcctgataagctttga

SEQ ID NO: 21 - DNA, 712 bp, Artificial Sequence
  the CTP-modified IGF-1, MOD-1301-2
gcggccgccatgggcaagatctccagcctgcctacccagctgttcaaatgctgtttctgcgactttctgaaggtgaaaatgcacacaatgtctagttcacacctgttctacctggccctgtgcctgctgacctttacatccagcgccactgctggaccagagaccctgtgcggagctgaactggtggacgcactgcagttcgtctgtggggataggggtttctactttaacaagccaacaggctatggatctagttcaaggcgggcccctcagactgggattgtcgacgagtgctgttttcggagctgcgatctgagacgcctggaaatgtattgtgcccctctgaagccagcaaaatcagcctccagctctagtaaggctccccctccaagtctgcctagcccttctagactgcctggaccatctgacactccaatcctgcctcagtcatccagctctaaagcaccccctccaagcctgcctagtccatcacgtctgcccggtccttctgataccccaattctgccccagagttcatccagcaaggcccctcccccatccctgccttctcctagcaggctgccaggcccatctgacacacctatcctgccacagtctagttcatccaaagctccccctccatctctgccctctcctagtagactgccaggaccctccgatacccccattctgcctcagtgataagctttga

SEQ ID NO: 22 - DNA, 628 bp, Artificial Sequence
  CTP-modified IGF-1, MOD-1301-3
gcggccgccatgggcaagatctcttcactgcccacccagttgttcaagtgctgtttctgcgactttctgaaggtgaagatgcacaccatgagtagctcacacctgttttatctggccctctgtctgctcaccttcacttctagtgccactgccggaccagaaaccctctgcggcgccgaactggtggacgcattgcagttcgtgtgcggagacaggggtttctactttaacaagccaacaggttacggctcctctagcagacgggctccccagaccggcatcgttgatgagtgctgttttaggtcctgtgacctcaggcgtctggagatgtattgcgctcccctgaaaccagccaagtctgcaagctcatcttccaaggcacctccaccttctctgccaagcccctctaggttgccaggcccttccgatacccccattttgcctcagtcatccagcagtaaggcaccacccccttccctgcctagcccttcaaggctgccaggccctagcgataccccaattctgccacagagctcaagctccaaagccccacctccctcactgccatccccttctcggctgccaggcccatccgatacccctatcttgccacagtgataagctttga

SEQ ID NO: 23 - DNA, 562 bp, Artificial Sequence
  CTP-modified IGF-1, MOD-1301-4
gcggccgccatggctaccggtagtaggactagcctgctcttggcatttggtctgctctgtctgccttggttgcaggagggcagtgcctccagctcctctaaagctcctccaccctctttgccaagcccctctagattgcctggtccatccgatactccaattctgcctcagggccctgagactttgtgcggcgctgaactggtggacgcactccagttcgtctgcggagacagaggcttctacttcaacaaacctactgggtatggttcttccagtcgtagggcaccacagacaggtatcgtggatgagtgttgcttcaggtcatgtgacctcaggcgtctggagatgtactgtgcaccactgaagcctgcaaaatccgcctcaagctccagtaaggctccacctccttcattgccaagcccttctcgtctgcccggtccaagcgacaccccaattctgccccagtcatcttccagcaaagccccacctccaagtctgcccagcccaagtcgactgcctggaccctctgatacccccatcctgccacagtgataagctttga

SEQ ID NO: 24 - DNA, 814 bp, Artificial Sequence
  CTP-modified IGF-1, MOD-1301-5
gcggccgccatggcaacaggtagtaggacttctttgctgctcgcctttggactgctgtgcctcccttggctgcaggagggctcagctagcagcagttccaaggctcctcccccatctctgccttcacccagcaggttgcccgggccatcagatactccaatcctcccccagtcttccagtagcaaagccccacctccctccctgccttcaccatccaggttgcctggtccaagcgatacacctatcctgccacagggacctgagacactctgtggtgcagagctggtggatgcattgcagtttgtttgcggcgacagagggttctacttcaacaagcctactggctatggttctagctccagaagagcaccacagaccggaatcgtggatgaatgctgcttccgttcctgcgacttgcgcagactggagatgtattgtgccccactcaaacctgctaagtccgccagttctagctccaaagctcctccaccctcactgcccagcccatcaaggctcccaggaccctcagatacccccattttgcctcagtctagctccagcaaggcacctccaccctctttgccctctccaagcagattgccaggtcctagtgacactcccatcctgcctcagtcaagctccagtaaagcccctccacctagcctcccatctcccagcagactgccaggtcctagcgatacacccatcttgccccagtcaagtagctccaaagctccaccccctagcctcccttcaccctctaggttgcctggcccatcagatacaccaattctcccacagtgataagctttga

SEQ ID NO: 25 - protein, 48 aa, Homo sapiens
MGKISSLPTQLFKCCFCDFLKVKMHTMSSSHLFYLALCLLTFTSSATA

SEQ ID NO: 26 - protein, 275 aa, Artificial Sequence
  CTP-HGH-CTP-CTP Polypeptide without a Signal Sequence
SSSSKAPPPSLPSPSRLPGPSDTPILPQFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSSSSKAPPPSLPSPSRLPGPSDTPILPQSSSSKAPPPSLPSPSRLPGPSDTPILPQ

SEQ ID NO: 27 - protein, 301 aa, Artificial Sequence
  CTP-HGH-CTP-CTP Polypeptide with the Signal Sequence
MATGSRTSLLLAFGLLCLPWLQEGSASSSSKAPPPSLPSPSRLPGPSDTPILPQFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSSSSKAPPPSLPSPSRLPGPSDTPILPQSSSSKAPPPSLPSPSRLPGPSDTPILPQ

SEQ ID NO: 28 - DNA, 903 bp, Artificial Sequence
  Encoding a CTP-modified hGH polypeptide
atggccaccggcagcaggaccagcctgctgctggccttcggcctgctgtgcctgccatggctgcaggagggcagcgccagctcttcttctaaggctccacccccatctctgcccagccccagcagactgccgggccccagcgacacacccattctgccccagttccccaccatccccctgagcaggctgttcgacaacgccatgctgagggctcacaggctgcaccagctggcctttgacacctaccaggagttcgaggaagcctacatccccaaggagcagaagtacagcttcctgcagaacccccagacctccctgtgcttcagcgagagcatccccacccccagcaacagagaggagacccagcagaagagcaacctggagctgctgaggatctccctgctgctgatccagagctggctggagcccgtgcagttcctgagaagcgtgttcgccaacagcctggtgtacggcgccagcgacagcaacgtgtacgacctgctgaaggacctggaggagggcatccagaccctgatgggccggctggaggacggcagccccaggaccggccagatcttcaagcagacctacagcaagttcgacaccaacagccacaacgacgacgccctgctgaagaactacgggctgctgtactgcttcagaaaggacatggacaaggtggagaccttcctgaggatcgtgcagtgcagaagcgtggagggcagctgcggcttcagctccagcagcaaggcccctcccccgagcctgccctccccaagcaggctgcctgggccctccgacacaccaatcctgccacagagcagctcctctaaggcccctcctccatccctgccatccccctcccggctgcctggcccctctgacacccctatcctgcctcag